(19) United States
(12) Patent Application Publication
(10) Pub. No.: US 2024/0261347 A1
(43) Pub. Date: Aug. 8, 2024

(54) PROBIOTIC FORMULATION AND DELIVERY

(71) Applicants: PROAGNI PTY LTD, New South Wales (AU); AUSTRIANOVA SINGAPORE PTE LTD, Singapore (SG)

(72) Inventors: Athol Victor Klieve, Queensland (AU); Fiona Eileen Soulsby, New South Wales (AU); Robert James Bell, New South Wales (AU); Lachlan Ian Campbell, New South Wales (AU); John Austin Dangerfield, Singapore (SG)

(21) Appl. No.: 17/796,553

(22) PCT Filed: Jan. 29, 2021
(86) PCT No.: PCT/AU2021/050064

§ 371 Date: Jul. 29, 2022

(30) Foreign Application Priority Data
Jan. 31, 2020 (AU) 2020900269
Jan. 31, 2020 (GB) 2001381.9

(51) Int. Cl. A61K 35/747; A23K 10/16; A23K 20/116; A23K 20/163; A23K 40/25; A23K 40/30; A61K 9/48; A61K 35/745; A61P 3/00

(52) U.S. Cl. CPC A61K 35/747; A23K 10/16; A23K 20/116; A23K 20/163; A23K 40/25; A23K 40/30; A61K 9/4816; A61K 9/4833; A61K 35/745; A61P 3/00

(57) ABSTRACT

The present disclosure relates generally to encapsulated strict obligate anaerobic bacteria, (e.g., Megasphaera eldesdenii and Ruminicoccus bromi), compositions comprising same, and the use of said capsules and/or compositions to deliver the strict obligate anaerobic bacteria to the gastrointestinal tract (GI) of an animal, such as a livestock animal (e.g., a ruminant or hindgut fermenter). The present disclosure relates to the encapsulation of strict obligate anaerobic bacteria which utilize lactic acid and/or starch and the use of those encapsulated bacteria in the field of animal health and nutrition. In particular examples, the present disclosure utilises porous capsules comprising surface pores with a molecular weight cut off between 50 and 200 kDa and wall comprising a complex formed from sodium cellulose sulphate and poly [dimethyldially-ammonium chloride].

### RELATED APPLICATION DATA

The present application claims priority from Australian Provisional Application No. 2020900269 filed on 31 Jan. 2020 and United Kingdom Patent Application No. 2001381.9 filed on 31 Jan. 2020, the full contents of which are incorporated herein by reference.

### TECHNICAL FIELD

The present disclosure relates generally to porous capsules comprising strict obligate anaerobic bacteria, compositions comprising same, and the use of said capsules and/or compositions to deliver strict obligate anaerobic bacteria to the gastrointestinal tract (GI) of an animal, such as a livestock animal (e.g., a ruminant or hindgut fermenter). In particular, the present disclosure relates to porous capsules comprising strict obligate anaerobic bacteria which utilize or metabolise lactic acid and/or starch and the use of those encapsulated bacteria in the field of animal health and nutrition. The present disclosure also relates to methods of improving shelf stability of probiotic formulations comprising strict obligate anaerobic bacteria.

### BACKGROUND

Roughage is an important dietary component for many livestock species, particularly ruminants and hindgut fermenters, which rely on the fiber content of roughage to stimulates mastication and, in the case of ruminants, rumination. This in turn stimulates the production of saliva which helps to buffer and balance acidity in the GI tract generated through the digestion of more readily fermentable carbohydrates, such as starches and sugars. However, in some cases, the large amount of energy required by animals in a production or performance setting is not met by a diet which is based solely or predominantly on roughage (which is relatively low in fermentable carbohydrates). In such circumstances, it may be desirable, or even necessary, to transition an animal from a diet which is roughage-based to an energy rich concentrate diet having a greater proportion of fermentable carbohydrate. Notwithstanding the benefits of an energy-rich diet in terms of production efficiency and/or animal performance, a concentrate rich diet without adequate roughage can cause metabolic dysregulation. For example, when certain livestock (such as ruminants and hindgut fermenters) are transitioned from a roughage-based diet to an energy rich concentrate diet which is low in structural carbohydrates, they may develop lactic acidosis.

Lactic acidosis is a metabolic disorder characterised by an accumulation of organic acids, especially lactic acid, in the GI tract (specifically the rumen and reticulum of ruminants, and the hind gut of hindgut fermenters). Lactic acidosis may be further categorised into sub-acute and acute acidosis. In the dairy industry, sub-acute rumen acidosis is a common and serious health and production problem because dairy cows are usually fed diets containing high levels of grains. Lactic acidosis is also a problem in segments of the beef industry where feedlotting is practiced. Sub-acute and acute rumen acidosis are simply different degrees of the same problem. Acute rumen acidosis is more severe and physiological functions may be significantly impaired. The affected animal is depressed and usually ataxic, off-feed, with dilated pupils and an elevated heart rate. Diarrhoea will be obvious and the animal may become recumbent and die within 2 to 5 days after the insult. Acute acidosis is characterised by a dramatic reduction in ruminal pH (below pH 5.0), a large increase in lactic acid concentration and a large decrease in protozoa. Sub-acute acidosis, on the other hand, is typically characterised by a reduction in pH within the range of 5.6 to 5.2. The symptoms of sub-acute rumen acidosis differ from that of acute acidosis and can be difficult to recognise within a large group. Herds with sub-acute rumen acidosis will typically present some or all of the following signs: laminitis, intermittent diarrhoea, poor appetite or cyclical feed intake, high herd cull rates for poorly defined health problems, poor body condition in spite of adequate energy intake, abscesses without obvious causes and hemoptysis (coughing of blood) or epistaxis (bleeding from the nose). Most of these signs are secondary to acidosis and most of them do not appear until weeks or months after the initial acidosis events. Contrary to feedlot cattle, dairy cows are kept for years and the management of acidosis is therefore of importance in increasing profits.

In almost all cases, lactic acidosis is caused by a gross imbalance between the numbers of lactic acid-producing bacteria (LAB) and lactic acid-utilising bacteria (LUB) in the GI tract, typically brought on by a sudden increase in the proportion of readily fermentable carbohydrates in the animal's diet and/or a lower proportion of roughage. This in turn increases the production of lactic acid in the GI tract. Further, a reduction in structural carbohydrates necessary for stimulating mastication and rumination reduce the animal's ability to buffer changes in acidity in the GI tract.

Traditionally, the transition from a high fibre, roughage-based diet to a concentrate-based diet takes 10-20 days of slow transition to ensure gastrointestinal upsets are minimised and reduce the incidence of lactic acidosis. A number of approaches exist for reducing this transition time, the majority of which rely on manipulating the microbial population in the GI tract during the transition in order to cope with increased levels of organic acids produced during fermentation. One particular approach which has attracted recent interest in both the beef and dairy cattle industries is the use of Direct-Fed Microbials (DFM), also known as “probiotics”. DFM is a term typically reserved for naturally occurring live microbes that can be supplemented orally to produce a beneficial health response in the host animal. A number of genera of live microorganisms, including bacteria, yeast and fungi, have been reported for use as DFM in domestic ruminants. However, most have been reported in an experimental context only and have not been commercialised for various reasons. In the context of lactic acidosis, a single DFM containing the LUB Megasphaera elsdenii (ME) has been approved by the US Food and Drug Administration (FDA) and marketed under the proprietary name Lactipro® (MS Biotech). ME is the major lactate-utilising organisms found in the rumen of adapted cattle fed high grain diets. However, the numbers of this organism are typically low in the rumens of cattle that have not been adapted to concentrate feed. When cattle are shifted from a high forage diet to a high concentrate diet, lactate-producing bacteria proliferate rapidly, whereas lactate-utilizing bacteria, such as ME lag and the number of bacteria is often insufficient to prevent lactic acidosis. Oral dosing of Megasphaera probiotic cultures has been shown to increase the population of lactate-utilizing bacteria, presumably thereby preventing accumulation of lactic acid. Lactipro®, marketed by MS Biotech, has been reported to reduce the transition time from a high roughage diet to a high concentrate diet in cattle by 50% whilst still maintaining adequate ruminal pH and preventing lactate accumulation. Unfortunately, however, most feed additives containing live bacterial cultures are unstable at ambient temperature, making it necessary to ship and store them under cool conditions. Even then, shelf life of these additives is typically short. In the case of Lactipro® which (at the time of filing this application) is a liquid product, shelf life is 14 days making it necessary to manufacture on demand. The strictly anaerobic nature of ME also makes Lactipro® (and any similar products) oxygen sensitive, necessitating administration via a drenching gun which is labour intensive and increases the dose volume needed for delivery.

In view of the above disadvantages with existing DFM products and approaches for management of lactic acidosis in livestock, particularly domesticated ruminants, there exists a need for improved DFM products and methods for manipulating populations of LAB and LUB in the GI tract of livestock.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure is based at least in part on the inventors' unexpected finding that encapsulation of strict obligate anaerobic bacteria in a porous microcapsule (also referred to herein as a “capsule”) improves stability of viable bacteria in the presence of oxygen (e.g., such as when exposed to normal atmospheric oxygen levels during administration to an animal) and extends the shelf-life of viable bacteria when stored in anaerobic conditions at ambient temperatures relative to corresponding bacterial cultures which are not encapsulated. This is surprising in view of the sensitivity of strict obligate anaerobic bacteria to oxygen, particularly at levels present in the atmosphere, and the porous nature of the capsules used in the encapsulation process. In this regard, the encapsulation material of the present disclosure had only previously been demonstrated for use with aerobic bacteria and certain anaerobic bacteria reported as being tolerant to low levels of oxygen. In contrast, the inventors have encapsulated two strict obligate anaerobes having low tolerance to oxygen, Megasphaera eldesdenii and Ruminicoccus bromii, in a porous capsule formed from a complex of sodium cellulose sulphate and poly[dimethyldially-ammonium chloride], having surface pores with a molecular weight cut off between 50 and 200 kDa. The inventors have also shown that freeze-drying the encapsulated bacteria improves their shelf life and stability when stored under anaerobic conditions at ambient temperatures, which has obvious benefits in terms of manufacture, distribution and end use. In animal trials conducted in cattle, the inventors have further demonstrated that oral administration of encapsulated freeze-dried Megasphaera elsdenii results in rapid colonisation of the animal's gastrointestinal tract, and that a single dose of the encapsulated, freeze-dried Megasphaera elsdenii was sufficient to facilitate rapid and sudden transition from a grass-based diet to a high concentrate finisher diet with no apparent impact on digestive health. Further, the rapid colonisation of the animal's gastrointestinal tract with Megasphaera elsdenii resulted in additional weight gain compared to control animals not administered the encapsulated bacteria.

Accordingly, the present disclosure provides a capsule comprising one or more strains of strict obligate anaerobic bacteria, wherein the capsule has a porous wall comprising surface pores with a molecular weight cut off between 50 and 200 kDa, wherein the porous wall comprises a complex formed from sodium cellulose sulphate and poly[dimethyldially-ammonium chloride].

In one example, the one or more strains of strict obligate anaerobic bacteria encapsulated in the capsule have improved stability in the presence of oxygen relative to a corresponding one or more strains of the bacteria not encapsulated in the capsule. For example, the one or more strains of strict obligate anaerobic bacteria remain viable for at least about 30 minutes in the presence of atmospheric oxygen levels when encapsulated. For example, the one or more strains of strict obligate anaerobic bacteria remain viable for at least about 45 minutes in the presence of atmospheric oxygen levels when encapsulated. For example, the one or more strains of strict obligate anaerobic bacteria remain viable for at least about 60 minutes in the presence of atmospheric oxygen levels when encapsulated. For example, the one or more strains of strict obligate anaerobic bacteria remain viable for at least about 90 minutes in the presence of atmospheric oxygen levels when encapsulated. For example, the one or more strains of strict obligate anaerobic bacteria remain viable for at least about 2 hours in the presence of atmospheric oxygen levels when encapsulated. For example, the one or more strains of strict obligate anaerobic bacteria remain viable for at least about 3 hours in the presence of atmospheric oxygen levels when encapsulated. In one examples the one or more strains of strict obligate anaerobic bacteria remain viable for greater than 3 hours in the presence of atmospheric oxygen levels when encapsulated.

In some examples, the capsule of the disclosure may be freeze dried, spray-dried or extruded. In one example, the capsule is freeze-dried. In one example, the capsule is spray-dried. In one example, the capsule is extruded.

In one example, at least one of the strains of strict obligate anaerobic bacteria in the capsule is a lactic acid-utilising bacteria (LUB). In one example, at least one of the strains is from the genus Megasphaera. For example, the capsule may comprise Megasphaera elsdenii e.g., the strain YE34.

Alternatively, or in addition, at least one of the strains of strict obligate anaerobic bacteria in the capsule is a starch-utilising bacteria. For example, at least one of the strains may be from the genus Ruminococcus. For example, the capsule may comprise Ruminicoccus bromii e.g., the strain YE282.

In one example, the capsule may comprise Megaphaera elsdenii and Ruminococcus bromii.

In one example, the capsule does not comprise bacteria from a genus selected from the group consisting of: Bifidobacterium, Bacteroides, Fusobacterium, Propionibacterium, Enterococcus, Lactococcus, Peptostrepococcus, Pediococcus, Leuconostoc, Weissella, Geobacillus, and Lactobacillus.

In each example of the capsule described herein, the bacteria may be suspended in the log phase of growth within the capsule.

In one example, the capsule contains at least 1×10³ CFU of the one or more strains of strict obligate anaerobic bacteria. For example, the capsule may contain at least 1×10⁴ CFU of the one or more strains of strict obligate anaerobic bacteria. For example, the capsule may contain at least 0.5×10⁵ CFU of the one or more strains of strict obligate anaerobic bacteria. For example, the capsule may contain at least 1×10⁵ CFU of the one or more strains of strict obligate anaerobic bacteria. For example, the capsule may contain at least 0.2×10⁶ CFU of the one or more strains of strict obligate anaerobic bacteria. For example, the capsule may contain at least 0.4×10⁶ CFU of the one or more strains of strict obligate anaerobic bacteria. For example, the capsule may contain at least about 1×10⁶ CFU of the one or more strains of strict obligate anaerobic bacteria.

In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 3 log. In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 2.5 log. In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 2 log. In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 1.5 log. In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 1 log.

In one example, the porous capsule contains at least about 0.2×10⁵ CFU of the strict obligate anaerobic bacteria one month after encapsulation following storage under anaerobic conditions at ambient temperatures. For example, the porous capsule may contain at least about 0.4×10⁵ CFU of the strict obligate anaerobic bacteria one month after encapsulation following storage under anaerobic conditions at ambient temperatures.

In one example, the porous capsule contains at least about 0.2×10⁵ CFU of the strict obligate anaerobic bacteria three month after encapsulation following storage under anaerobic conditions at ambient temperatures. For example, the porous capsule may contain at least about 0.4×10⁵ CFU of the strict obligate anaerobic bacteria three month after encapsulation following storage under anaerobic conditions at ambient temperatures.

In one example, the porous capsule contains at least about 0.1×10⁵ CFU of the strict obligate anaerobic bacteria eight month after encapsulation following storage under anaerobic conditions at ambient temperatures. For example, the porous capsule may contain at least about 0.2×10⁵ CFU of the strict obligate anaerobic bacteria eight month after encapsulation following storage under anaerobic conditions at ambient temperatures.

In each of the foregoing examples describing CFUs per capsule following storage at ambient temperatures, the capsule may be stored at a temperature of up to about 35° C. For example, the capsule may be stored at a temperature of up to about 4° C. to about 35° C. For example, the capsule may be stored at a temperature of up to about 4° C. to about 22° C. For example, the capsule may be stored at about room temperature. In other examples, the capsule may be stored at a temperature of about 4° C. or below.

The present disclosure also provides a composition comprising one or more capsules described herein.

In some example, the composition may further comprise one or more carriers. In some example, the composition may comprise an oil carrier which is suitable for ingestion by an animal e.g., a livestock species described herein. For example, the composition may comprise an oil carrier which has a low oxygen diffusion rate.

In some example, the composition is an animal feed additive.

The composition may be provided in a dry from or a liquid form. In one example, the composition is provided in a dry form. In another example, the composition is provided in a liquid form.

The composition may be provided in a dosage form comprising at least about 1×10⁵ to about 1×10¹² CFU of the strict obligate anaerobic bacteria. For example, the composition may be provided in a dosage form comprising at least about 1×10⁶ to about 1×10¹¹ CFU of the strict obligate anaerobic bacteria. For example, the composition may be provided in a dosage form comprising at least about 1×10⁷ to about 1×10¹⁰ CFU of the strict obligate anaerobic bacteria. For example, the composition may be provided in a dosage form comprising at least about 1×10⁷ to about 1×10⁹ CFU of the strict obligate anaerobic bacteria. In one example, the composition is provided in a dosage form comprising at least about 1×10⁶ CFU of the at least one strict obligate anaerobic bacteria.

The composition of the disclosure may be packaged under anaerobic conditions. Accordingly, in some examples, the present disclosure provides a capsule or composition as described herein packaged in a container under anaerobic conditions.

Accordingly, the present disclosure also provides a method of increasing a population of a strict obligate anaerobic bacteria in the gastrointestinal tract of an animal, comprising administering a capsule of the disclosure or a composition comprising same as described herein to the animal.

By increasing the population of the strict obligate anaerobic bacteria in the gastrointestinal tract of the animal, the method of the disclosure may achieve one or more of the following:
(i) facilitate adaptation of the animal to a diet having a relatively higher amount of fermentable carbohydrates;
(ii) reduce accumulation of organic acid in the gastrointestinal tract of the animal;
(iii) improve starch utilisation in the gastrointestinal tract of the animal;
(iv) prevent or treat lactic acidosis, or one or more associated conditions or clinical symptoms thereof, in the animal;
(v) induce satiety in the animal;
(vi) control or modulate food intake by the animal;
(vii) improve lactate utilization in the gastrointestinal tract of the animal;
(viii) improve one or more production traits selected from milk production, feedlot performance, growth rate, rate of gain, finishing time and feed conversion efficiency;
(ix) lower the chance of digestive morbidity and/or mortality of the animal; and/or
(x) improve tolerance of an animal to a diet which is relatively higher in fermentable carbohydrates and/or lower in structural carbohydrates, without development of lactic acidosis.

Accordingly, the method of increasing a population of the strict obligate anaerobic bacteria in the gastrointestinal tract of the animal as described herein may be performed for the purpose of achieving one or more of the above outcomes. The present disclosure also provides a method of improving the stability of a strict obligate anaerobic bacteria when stored in the presence of atmospheric oxygen levels, said method comprising encapsulating the bacteria in a capsule having a porous wall comprising surface pores with a molecular weight cut off between 50 and 200 kDa, wherein the porous wall comprises a complex formed from sodium cellulose sulphate and poly[dimethyldially-ammonium chloride]. For example, the stability of the strict obligate anaerobic bacteria in the presence of atmospheric oxygen levels when encapsulated is improved relative to a corresponding bacteria which is not encapsulated in accordance with the present disclosure.

In one example, one or more of the encapsulated strict obligate anaerobic bacteria remain viable for at least about 30 minutes in the presence of atmospheric oxygen levels. In one example, one or more of the encapsulated strict obligate anaerobic bacteria remain viable for at least about 45 minutes in the presence of atmospheric oxygen levels. In one example, one or more of the encapsulated strict obligate anaerobic bacteria remain viable for at least about 60 minutes in the presence of atmospheric oxygen levels. In one example, one or more of the encapsulated strict obligate anaerobic bacteria remain viable for at least about 90 minutes in the presence of atmospheric oxygen levels. In one example, one or more of the encapsulated strict obligate anaerobic bacteria remain viable for at least about 2 hours in the presence of atmospheric oxygen levels. In one example, one or more of the encapsulated strict obligate anaerobic bacteria remain viable for at least about 3 hours in the presence of atmospheric oxygen levels. In one example, one or more of the encapsulated strict obligate anaerobic bacteria remain viable for longer than 3 hours in the presence of atmospheric oxygen levels.

The present disclosure also provides a method of improving stability of a strict obligate anaerobic bacteria when stored under anaerobic conditions at ambient temperatures, said method comprising encapsulating the bacteria in a capsule having a porous wall comprising surface pores with a molecular weight cut off between 50 and 200 kDa, wherein the porous wall comprises a complex formed from sodium cellulose sulphate and poly[dimethyldially-ammonium chloride]. For example, the stability of the strict obligate anaerobic bacteria stored under anaerobic conditions at ambient temperatures is improved when encapsulated relative to a corresponding bacteria which is not encapsulated in accordance with the present disclosure.

In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 3 log. In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 2.5 log. In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 2 log. In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 1.5 log. In one example, the number of cells of the one or more strains of strict obligate anaerobic bacteria that remain viable within the capsule after 30 days (e.g., at least one month or at least 2 months or at least 3 months or at least 4 months) at ambient temperature is reduced by less than or equal to 1 log.

In one example, the porous capsule contains at least about 0.4×10⁵ CFU of the strict obligate anaerobic bacteria one month after encapsulation following storage under anaerobic conditions at ambient temperatures. In one example, the porous capsule contains at least about 0.4×10⁵ CFU of the strict obligate anaerobic bacteria three month after encapsulation following storage under anaerobic conditions at ambient temperatures. In one example, the porous capsule contains at least about 0.2×10⁵ CFU of the strict obligate anaerobic bacteria eight month after encapsulation following storage under anaerobic conditions at ambient temperatures.

In each of the foregoing examples describing CFUs per capsule following storage at ambient temperatures, the capsule may be stored at an ambient temperature of up to about 35° C. For example, the capsule may be stored at a temperature of up to about 4° C. to about 35° C. For example, the capsule may be stored at a temperature of up to about 4° C. to about 22° C. For example, the capsule may be stored at about room temperature. In other examples, the capsule may be stored at a temperature of about 4° C. or below.

The present disclosure also provides for use of one or more capsules of the disclosure in the manufacture of a medicament for preventing or treating lactic acidosis, or one or more associated conditions or clinical symptoms thereof, in an animal, wherein the medicament is formulated for administration to the gastrointestinal tract of the animal.

In any of the foregoing examples, a condition associated with lactic acidosis is selected from the group consisting of rumenitis, lactic acidosis induced laminitis, lactic acidosis induced bloat, polioencaphomelacia (PEM), colic, gastric ulcers, dehydration and liver abscesses.

In any of the foregoing examples, a clinical symptom of lactic acidosis is selected from the group consisting of reduced feed intake, reduced feed-conversion efficiency, weight loss, lameness, diarrhea, dehydration, reduced physical performance, slow recovery from exercise, crib-biting, wind-sucking and weaving behaviour.

The acidosis may be acute acidosis or subacute acidosis. In one example, the acidosis is acute acidosis. In another example, the acidosis is subacute acidosis.

In some examples, the animal described herein is a livestock species. For example, the livestock species may be a ruminant species e.g., cattle, buffalo, sheep, goat, deer or camelid. In another example, the livestock species is a monograstric species (e.g., a horse, pig or poultry). The monogastric livestock species may be a hingut fermenter e.g., a horse.

In some example, administration of the encapsulated obligate anaerobic bacteria to the gastrointestinal tract of the animal maintains a stable pH in the gastrointestinal tract of the animal. In one example, the animal is a ruminant and administration of the encapsulated bacteria to the gastrointestinal tract of the animal increases pH of the rumen and/or maintains pH of the rumen above about 5.5. In another example, the animal is a hindgut fermenter and administration of the encapsulated bacteria to the gastrointestinal tract of the animal increases pH of the hindgut and/or maintains pH of the hindgut above about 5.5. In another example, the animal is a hindgut fermenter and administration of the encapsulated bacteria to the gastrointestinal tract of the animal increases pH of the hindgut and/or maintains pH of the hindgut above about 5.5.

In some examples, the strict obligate anaerobic bacteria may a lactic acid-utilising bacteria (LUB). In another example, the strict obligate anaerobic bacteria may a starch utilising bacteria. A strict obligate anaerobic bacteria which utilises lactic acid may be selected from the genus Megasphaera. In one example, the lactic acid utilising bacteria is Megasphaera eldesdenii e.g., the strain YE34.

In another example the strict obligate anaerobic bacteria may be a starch utilising bacteria. A starch utilising bacteria may be selected from the genus Ruminicoccus. In one example, the starch utilising bacteria is Ruminicoccus bromii e.g., the strain YE282.

In another example, a porous capsule described herein may comprise a lactic acid utilising bacteria as described herein and a starch utilising bacteria as described herein. For example, the porous capsule of the disclosure may comprise Megasphaera eldesdenii and Ruminicoccus bromii.

In some example, administration of the encapsulated bacteria to the gastrointestinal tract of the animal improves lactate and/or starch utilization.

In some examples of the method or use described herein, at least about 1×10⁵ to about 1×10¹² CFU of the strict obligate anaerobic bacteria is administered to the gastrointestinal tract of the animal via a capsule of the disclosure or a composition comprising same as described herein. For example, at least about 1×10⁶ to about 1×10¹¹ CFU of the or each strict obligate anaerobic bacteria is administered to the gastrointestinal tract of the animal via a capsule of the disclosure or a composition comprising same as described herein. For example, at least about 1×10⁷ to about 1×10¹⁰ CFU of the or each strict obligate anaerobic bacteria is administered to the gastrointestinal tract of the animal via a capsule of the disclosure or a composition comprising same as described herein. For example, at least about 1×10⁷ to about 1×10⁹ CFU of the or each strict obligate anaerobic bacteria is administered to the gastrointestinal tract of the animal via a capsule of the disclosure or a composition comprising same as described herein. In some examples, at least about 1×10⁶ CFU of the or each strict obligate anaerobic bacteria is administered to the gastrointestinal tract of the animal via a capsule of the disclosure or a composition comprising same as described herein.

The encapsulated obligate anaerobic bacteria may be administered to the animal at any time e.g., prior to feeding an animal, at the same time as feeding an animal, or post feeding an animal. The encapsulated strict obligate anaerobic bacteria may be administered to the animal with feed e.g., as a feed supplement, or separate to feed.

In accordance with a method of the disclosure which reduces accumulation of organic acid (e.g., lactic acid) in the gastrointestinal tract of an animal and/or facilitates adaptation of the animal to a diet having a relatively higher amount of fermentable carbohydrates and/or which prevents or treats lactic acidosis, the encapsulated obligate anaerobic bacteria may be administered to the animal prior to, at the same time as, or following an increase an amount of fermentable carbohydrate in the animal's diet. In one example, the encapsulated strict obligate anaerobic bacteria is administered to the animal prior to increasing an amount of fermentable carbohydrate in the animal's diet. In one example, the encapsulated strict obligate anaerobic bacteria is administered to the animal prior at the same time as increasing an amount of fermentable carbohydrate in the animal's diet (e.g., the encapsulated obligate anaerobic bacteria may be provided with the food as a feed supplement). In one example, the encapsulated strict obligate anaerobic bacteria is administered to the animal after increasing an amount of fermentable carbohydrate in the animal's diet (e.g., such as in response to one or more symptoms of acidosis presenting in the animal).

The present disclosure also provides a method of producing a capsule of the disclosure in which the strict obligate anaerobic bacteria are encapsulated, the method comprising:
(i) culturing one or more strict obligate anaerobic bacteria in an anaerobic media;
(ii) encapsulating the bacteria in a capsule having a porous wall comprising surface pores with a molecular weight cut off between 50 and 200 kDa, wherein the porous wall comprises a complex formed from sodium cellulose sulphate and poly[dimethyldially-ammonium chloride];
(iii) performing one or more step which suspend growth of bacteria.

In one example, the method comprises cultivating the encapsulated bacteria in an anaerobic media prior to performing step (iii).

In one example, the anaerobic media comprises peptone, meat extract, yeast extract, glucose, tween-80, K₂HPO₄, sodium acetate, (NH₄)₂ citrate, MgSO₄-7H₂O and MnSO₄—H₂O. In accordance with an example in which the strict obligate anaerobic bacteria is a lactic acid-utilising bacteria, the cultivation media may be anaerobic modified de Man, Rogosa and Sharpe (MRS) media. In accordance with an example in which the strict obligate anaerobic bacteria is a starch-utilising bacteria, the cultivation media may be anaerobic maltose media modified with rumen fluid or anaerobic basal Yeast extract-Casitone-Fatty Acids (YCFA) medium supplemented with a starch or a sugar source (e.g., glucose). The one or more step which suspend the growth of bacteria may comprise any one of freeze-drying, spray-drying or extrusion. In one example, the method comprises performance of a freeze-drying step to suspend growth of bacteria. In one example, the method comprises performance of a spray-drying step to suspend growth of bacteria. In one example, the method comprises performance of an extrusion step to suspend growth of bacteria.

In one example, each porous capsule produced by the method contains at least 1×10³ CFU of the strict obligate anaerobic bacteria. For example, each porous capsule produced by the method may contain at least 1×10⁴ CFU of the strict obligate anaerobic bacteria. For example, each porous capsule produced by the method may contain at least 0.5×10⁵ CFU of the strict obligate anaerobic bacteria. For example, each porous capsule produced by the method may contain at least 1×10⁵ CFU of the strict obligate anaerobic bacteria. For example, each porous capsule produced by the method may contain at least 0.2×10⁶ CFU of the strict obligate anaerobic bacteria. For example. each capsule may contain at least 0.2×10⁶ CFU of bacteria. For example, each porous capsule produced by the method may contain at least 0.4×10⁶ CFU of the strict obligate anaerobic bacteria. For example, each porous capsule may contain at least about 1×10⁶ CFU of the one or more strains of strict obligate anaerobic bacteria.

In one example, the bacteria are suspended in the log phase of growth.

Encapsulating the strict obligate anaerobic bacteria in the porous capsule according to the method described herein increase stability of viable bacteria in the presence of oxygen e.g., normal atmospheric levels of oxygen, compared to a corresponding bacteria which has not been encapsulated. In one example, one or more of the encapsulated strict obligate anaerobic bacteria remain viable for at least about 30 minutes in the presence of oxygen. In one example, one or more of the encapsulated strict obligate anaerobic bacteria remain viable for at least about 45 minutes in the presence of oxygen. In one example, one or more of the encapsulated strict obligate anaerobic bacteria remain viable for at least about 60 minutes in the presence of oxygen. In one example, one or more of the encapsulated strict obligate anaerobic bacteria remain viable for at least about 90 minutes in the presence of oxygen. In one example, one or more of the encapsulated strict obligate anaerobic bacteria remain viable for at least about 2 hours in the presence of oxygen. In one example, one or more of the encapsulated strict obligate anaerobic bacteria remain viable for at least about 3 hours in the presence of oxygen. In some examples, one or more of the encapsulated strict obligate anaerobic bacteria remain viable for longer than 3 hours in the presence of oxygen.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1. Microscopic analysis of encapsulated M. eldesnii. The encapsulated M. eldesnii were analysed under 40× and 100× magnification on Days 0, 1 and 2 post encapsulation. The capsules were of standard size and uniform shape. The M. eldesnii filled up the capsules and the capsules were full by Day 2 post-encapsulation.

FIG. 2. Microscopic analysis of encapsulated R. bromii. The encapsulated R. bromii were analysed under 40×, 100×, 200× and 400× magnification on Days 1, 2 and 3 post encapsulation. At Day 1 post encapsulation, filamentous bacteria were seen inside the capsules. Capsules were 30% full with bacterial filaments. At Day 2 post encapsulation, capsules were observed to be 60% full with long filaments. At Day 3 post encapsulation, most capsules were 80% full with long filaments.

FIG. 3. Freeze-dried encapsulated M. eldesni. A. Vials of freeze-dried encapsulated M. eldesnii. B. Microscopic analysis of rehydrated freeze-dried encapsulated M. eldesnii.

FIG. 4. Analysis of rehydrated freeze-dried encapsulated R. bromii. A. Prestoblue assay of rehydrated freeze-dried encapsulated R. bromii. Rehydrated RB capsules were incubated in Maltose media with Rumen for up to 3 days. Capsule viability was monitored by Prestoblue daily using Maltose media without Resazurin, Rumen Fluid and L-cysteine. The RFU reading peaked on Day 1 but dropped on Day 2 and 3 of culture. B. Microscopic analysis of rehydrated R. bromii. The rehydrated R. bromii capsules were incubated in Maltose media with Rumen. The bacteria grew with some completely filling up the capsules by Day 2 of culture.

FIG. 5. Growth curve of R. bromii. The freeze-dried encapsulated R. bromii was decapsulated and put into Maltose media to monitor growth. 0.1 ml, 0.2 ml, 0.5 ml RB free bacteria were inoculated into Maltose media as a free bacteria control. The OD₆₀₀ₙₘ of both free bacteria and decapsulated bacteria was measured at the following timepoints: 22 hours, 24 hours, 26 hours, 27 hours, 28 hours, 29.5 hours and 48 hours showing that the bacteria are viable and growing.

FIG. 6. Stability of freeze-dried encapsulated M. elsdenii. The stability of freeze-dried encapsulated M. elsdenii was measured over 20 months at ambient storage. A loss of 1-log was observed in the first month.

FIG. 7. Agarose gel electrophoresis of gDNA extractions from M. eldesnii. Extracted gDNA (5 μL) run on a 1% agarose gel electrophoresis in TAE buffer visualised under UV with GelRed stain. Top row: M-DNA marker; Lanes 1-3 modified RF+ medium; Lanes 4-6 rumen fluid from steer #1990; Lanes 7-9 ME 0 h; Lanes 10-12 Con 0 h; Lanes 13-15 ME 1 h; Lanes 16-18 Con 1 h; M-DNA marker. Bottom row: M-DNA marker; Lanes 1-3 ME 2 h; Lanes 4-6 Con 2 h; Lanes 7-9 ME 3 h; Lanes 10-12 Con 3 h; Lanes 13-15 ME 4 h; Lanes 16-18 Con 4 h; M-DNA marker.

FIG. 8. Agarose gel electrophoresis of gDNA extractions from M. eldesnii. Extracted gDNA from 1.0 mL samples run on a 1% agarose gel electrophoresis in TAE buffer visualised under UV with GelRed stain. Top row M-DNA marker; Lanes 1-3 ME 5 h; Lanes 4-6 Con 5 h; Lanes 7-9 ME 6 h; Lanes 10-12 Con 6 h; Lanes 13-15 ME 19 h; Lanes 16-18 Con 19 h; M-DNA marker. Bottom row M-DNA marker; Lanes 1-3ME 24 h; Lanes 4-6 Con 24 h; Lanes 7-9 ME 48 h; Lanes 10-12 Con 48 h; M-DNA marker.

FIG. 9. qPCR assay to quantify the numbers of M. eldesnii in the feedlot ration. Population levels of M. elsdenii (cells/mL) present in three replicates of two treatments—Control (no probiotic) and ME (addition of one vial of encapsulated M. elsdenii) across a 48 h period.

FIG. 10. Batch culture pH at each sampling point for the M. elsdenii and control replicates across the 48 h of incubation. The pH of the Control replicates did not drop to acute acidosis level (pH<5.0) or subacute acidosis levels (pH<5.8) but were seen to be lower than the M. elsdenii treatment groups from 19 h onwards.

### DETAILED DESCRIPTION

### General Techniques and Definitions

Unless specifically defined otherwise, all technical and scientific terms used herein shall be taken to have the same meaning as commonly understood by one of ordinary skill in the art (e.g. in animal nutrition, feed formulation, microbiology, livestock management).

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

As used herein, the terms “preventing”, “prevent”, or “prevention” include administering an effective amount of a composition, supplement or feed to an animal e.g., a livestock species, sufficient to stop or hinder the development of at least one symptom of lactic acidosis, or an associated condition or symptom thereof.

The term “about” is used herein to mean approximately. When the term “about” is used in conjunction with a numerical range, it modifies that range by extending the boundaries above and below the recited numerical values. In general, the term “about” is used herein to modify a numerical value above and below the stated value by 10%, up or down (higher or lower).

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features. Thus, each feature of any particular aspect or embodiment of the present disclosure may be applied mutatis mutandis to any other aspect or embodiment of the present disclosure.

The present disclosure is not to be limited in scope by the specific embodiments described herein, which are intended for the purpose of exemplification only. Functionally equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Encapsulation

The strict obligate anaerobic bacteria disclosed herein are encapsulated in capsules having a porous capsule wall. As used herein, the term “encapsulated” refers to its conventional meaning within the art. Thus, encapsulation as used herein refers to the process of forming a continuous coating around an inner matrix or cell that is wholly contained within the capsule wall as a core of encapsulated material. Encapsulation is to be distinguished from “immobilisation” which refers to the trapping of material such as cells within or throughout a matrix. In contrast to encapsulation, immobilisation is a random process resulting in undefined particle size where a percentage of immobilised elements will be exposed at the surface. Encapsulation or microencapsulation (both terms are used herein interchangeably) helps to separate a core material from its environment, thereby improving its stability and extending the shelf-life of the core material. The structure formed by the microencapsulation agent around the core substance is known as the wall or shell. The properties of the wall system are typically designed to protect the core material and to potentially release the core material under specific conditions while allowing small molecules to pass in and out of the porous capsule wall (that acts as a membrane). The capsules may, for example, range from submicron to several millimetres in size and can be of different shapes.

The porous capsule used in the present disclosure has a wall comprised of a complex formed from cellulose sulphate and poly[dimethyldiallyl-ammonium chloride] (pDADMAC). The cell microencapsulation technology used herein is based on the use of sodium cellulose sulphate which may be produced either by homogenously or heterogeneously sulphated cellulose. Methods of encapsulation with this technology are described in PCT publication no. WO2012/101167, which is herein incorporated by reference.

The pDADMAC used in the methods and capsules disclosed herein is as described in Dautzenberg et al., (1999) Ann. N. Y. Acad. Sci., 875:46-63. In Dautzenberg et al., (1999b), it was disclosed that the optimum mechanical strength of the capsule wall can be achieved with pDADMAC of about 20 kDa. Capsules produced this way are characterised as having pores large enough to allow passage of proteins or monoclonal antibodies, according to a size of at least 80 kDa or even up to 150 kDa. The dependency of pore size and the size of the pDADMAC used herein has been described in Dautzenberg et al., (1999) Journal of Membrane Science, 162(1-2): 165-171 (Dautzenberg et al., (1999a)). It is clear that a lower molecular weight of the pDADMAC results in a larger pore size. The full contents of Dautzenberg et al., (1999a and 1999b) are incorporated by reference herein in their entirety.

In one example, the porous capsule wall comprises a polyelectrolyte complex formed from the counter-charged poly electrolytes cellulose sulphate and poly[dimethyldiallyl-ammonium chloride].

The capsules may be in the form of spheric microcapsules with a diameter of between 0.01 and 5 mm, or between 0.05 and 3 mm, or between 0.01 and 1 mm, or between 0.2 mm and 1.2 mm. The capsules have a porous capsule wall. The microcapsules are characterized as to comprise surface pores. The surface pore size of the porous capsule wall may be between 80 nm and 150 nm, to allow the enzymes to pass. The surface pores of the porous capsule wall have a molecular weight cut off (MWCO) of between 50 and 200 kDa, or between 60-150 kDa, or between 60 and 100 kDa.

The production of cellulose sulphate of sufficient quality has been described in WO/2006/095021 (US 20090011033). The cellulose sulphate may be between 100-500 kDa, or between 200-400 kDa, or between 250-350 kDa.

The preparation and synthesis of cellulose sulphate capsules has been thoroughly described in DE 40 21 050 A1. Methods for a comprehensive characterization of cellulose sulphate capsules have been extensively dealt with in Dautzenberg et al, (1993) Biomat. Art. Cells & Immob. Biotech., 21(3):399-405. Other cellulose sulphate capsules have been described in GB 2 135 954. The properties of the cellulose capsules, i.e. the size, the pore size, wall thickness and mechanical properties depend upon several factors such as for example physical circumstances wherein the capsules have been prepared, viscosity of precipitation bath, ion strength, temperature, rapidity of addition of cell/cellulose sulphate suspension, constitution of cellulose sulphate, as well as other parameters have been previously described.

Generally, in order to form the capsules of the disclosure, the sodium cellulose sulphate is brought in contact with an aqueous pDADMAC solution. Alternatively, poly[dimethyldiallyl-ammonmm chloride] (pDADMAC or also referred to as PDMDAAC) may be prepared via radical polymerization of dimethyl-diallyl-ammonium chloride. Mansfeld and Dautzenberg suggest to use a 1.2% (w/v) solution of PDMDAAC (pDADMAC) in destilled water. Zhang et al., (2005) Journal of Membrane Science, 255(1-2):89-98 describe the use of a pDADMAC with a molecular weight of 200,000-350,000 Da, whereas Dautzenberg suggests a pDADMAC of a molecular weight of 10,000-30,000 Da.

In WO/2006/095021, a method has been described that results in cellulose sulphate samples of sufficient quality. In this process a reaction mixture of n-propanol and sulphuric acid served as sulphating medium and agent.

Sodium cellulose sulphate serves as polyanion and pDADMAC serves as a polycation. The NaCS solution is used to build the capsule core and the pDADMAC solution as a precipitation bath delivering the second reaction component for PEC formation at the surface of the droplets, thus forming the capsules by covering the droplets with a solid membrane.

Any commercially available encapsulating machine may be used to form microcapsules. Such an encapsulator will typically include a perfussor drive which pushes a NaCS solution with defined velocity through a nozzle and thus generates a continuous liquid flow. The liquid flow is forced to oscillate by a pulsation unit, where the superimposed oscillation causes the break-off of the outlet liquid stream or jet into beads of equal volume. In order to improve the mono-dispersibility of the beads and at the same time to reduce coalescence, an electric field is provided under the nozzle outlet in such an encapsulator. Electrostatic charging in the free phase causes a repulsion of the individual beads, so that an aggregation of the individual beads up to entry into the complex- forming bath is substantially prevented.

The sodium cellulose sulphate may be produced by the homogenously sulphating method starting with cellulose linters. Alternatively, heterogenously sulphated cellulose may be used as described in Dautzenberg et al., (1999b) which results in the formation of capsules with large pores, of at least 80 kDa.

The spheric beads formed in this manner may be dropped into a complex-forming bath, within which at the outer membrane of the capsule is formed around the capsule by electrostatic interaction, for example between the NaCS and a pDADMAC solution. Under constant stirring, the capsules remain in this system until reaching a desired hardening degree in the corresponding container and are then available for further processing.

In the absence of an encapsulator or other airjet droplet generator system, a syringe with a 0.2 to 1.0 mm inner diameter needle possibly with a suitable syringe pump extrusion system may be used. Alternatively, a pasteur pipette e.g. with an inner diameter of 1.5 mm may also be used.

The resulting capsules may have a pore size large enough to allow macromolecules up to 80 kDa or even up to 150 kDa to pass. Capsules produced that way have been reported to have pore sizes large enough to release antibodies through these pores which are produced from hybridoma cells within these capsules. The cellulose sulphate encapsulation technology described by Dautzenberg et al., (1999b) has also been employed to test whether in vivo production of a neutralising monoclonal antibody could protect mice against Fr-CasE retrovirus (Pelegrin et al., (2000) Human Gene Therapy, 11:1407-1415). These results demonstrated that the capsules have pores large enough to allow a monoclonal antibody to pass through. Equally, the resulting capsules will have a pore size large enough for nutrients and bacterial food sources to enter the capsule.

It is understood, however, that the substances and methods disclosed herein need not be limited to the use of the specific ingredients described herein. Instead, the use of ingredients purchased from other sources or ingredients, produced by methods such as described above may also be used.

### Microorganisms and Culturing of Microorganisms

Microorganisms may be classified into different groups according to their requirement for oxygen. For example, “aerobes”, “aerobic bacteria”, “obligate aerobes”, or similar, are those bacteria whose metabolic pathways require oxygen to produce ATP. “Facultative anaerobes” are those bacteria which make ATP by aerobic respiration if oxygen is present, but are capable of switching to fermentation or anaerobic respiration if oxygen is absent. By contrast, “obligate anaerobes”, are microorganisms that cannot produce ATP in the presence of excessive oxygen because they utilize metabolic pathways which rely on enzymes that react with oxidants. Instead, obligate anaerobes rely on anaerobic respiration or fermentation to produce ATP and are killed by normal atmospheric concentrations of oxygen (20.95%). However, even within the category of “obligate anaerobe”, the level of oxygen tolerance or “aerotolerance” varies between species. For example, tolerance of obligate anaerobes to oxygen typically ranges between <0.5% and 8% O₂. In this regard, there is a spectrum of aerotolerance and some species of obligate anaerobe are capable of maintaining viability (and even growing) under conditions of partial aeration. For example, Shimamura et al., (1992) Journal of Dairy Science, 75(12):3296-3306 and Gonzalez-Cervantes et al., (2004) Applied Microbiology and Biotechnology, 65:606-610, describe aerotolerance of several Bifidobacterium species. In this regard, obligate anaerobes can be subdivided into two sub-categories based on the percentage of oxygen that can prove toxic: “strict obligate anaerobes” which will not survive if there is >0.5% oxygen in the environment, and “moderate obligate anaerobes” which can survive and even grow if there is >0.5%, and as much as 2% to 8% oxygen in the environment. It is generally understood that the protective mechanism that allows certain species of obligate anaerobes to avoid oxidative damage and survive in the presence of partial aeration is the ability to produce two enzymes, superoxide dismutase (SOD) and catalase. SOD is believed to be indispensable to all aerobes. The particularly low levels or even lack of SOD among “strict obligate anaerobes” is believed to be the reason for their oxygen intolerance. Accordingly, reference herein to “strict obligate anaerobes”, “strict obligate anaerobic bacteria”, or similar, shall be understood to mean those obligate anaerobic bacteria which have a low tolerance to oxygen or which are intolerant to oxygen altogether. For example, strict obligate anaerobic bacteria of the present disclosure will generally lose viability in the presence of >0.5% O₂. In any one of the foregoing examples, a strict obligate anaerobe may be one that produces little or no SOD, and which therefore reduces relatively large quantities of oxygen when exposed thereto.

The strict obligate anaerobic bacteria of the disclosure may be bacteria which are beneficial to an animal e.g., a livestock animal, when administered e.g., orally to the gastrointestinal tract. Examples of strict obligate anaerobic microorganisms include, but are not limited to, bacteria from the genus Clostridum, Meghasphaera and Ruminococcus. The strict obligate anaerobic bacteria of the disclosure may be bacteria from the genus Meghasphaera. In one example, the strict obligate anaerobic bacteria is Megasphaera elsdenii. Alternatively, or in addition, the strict obligate anaerobic bacteria may be bacteria from the genus Ruminococcus. In one example, the strict obligate anaerobic bacteria is Ruminococcus bromii.

Megasphaera elsdenii is a strict obligate anaerobe which typically inhabits the rumen of ruminant animals, such a cattle and sheep, although it can also be cultured from the intestinal contents of pigs and humans. M. elsdenii can utilize lactate to produce butyrate, a key volatile fatty acid often implicated in driving calf rumen development. For this reason it is classified as a lactic acid-utilizing bacteria (or LUB). By administering encapsulated bacteria, such as M. elsdenii and other LUBs, to the gastrointestinal tract of an animal, the methods and uses of the present disclosure seek to take advantage of the ability of these bacteria to utilize lactic acid and thereby prevent (an unhealthy) accumulation of lactic acid in the gastrointestinal tract. It will be appreciated by those skilled in the art that any M. elsdenii strain may be used. In one example, the M. elsdenii strain is YE34.

Ruminococcus bromii is a strict obligate anaerobic bacteria which is typically found in the gastrointestinal tract of monogastrics and ruminants. It is an amylolytic bacteria which has the ability to inhabit high starch environments (such as in the rumen) and break down starches (including resistant starches). By administering encapsulated bacteria, such as R. bromii to the gastrointestinal tract of an animal, the methods and uses of the present disclosure seek to take advantage of the ability of these bacteria to break down resistant starches and fibers, thereby improving starch utilization within the animal. It will be appreciated by those skilled in the art that any R. bromii strain may be used. In one example, the R. bromii strain is YE282.

In some examples, the anaerobic bacteria is not a bacteria selected from the group consisting of: Bifidobacterium, Bacteroides, Fusobacterium, Propionibacterium, Enterococcus, Lactococcus, Peptostrepococcus, Pediococcus, Leuconostoc, Weissella, Geobacillus, and Lactobacillus.

It will be appreciated that the bacterial strain used in the methods and uses of the present disclosure may be a non-genetically modified bacterium or the bacterial strain used may be a genetically modified bacterium. In one example, the bacterial strain is a non-genetically modified bacterium. In another example, the bacterial strain is a genetically modified bacterium. The bacterial strain may be genetically modified to comprise one or more nucleic acid molecule(s) encoding at least one heterologous antigen or a functional fragment thereof. It will be appreciated by those skilled in the art that the bacterium may be genetically modified by any method known in the art.

In order for the strict obligate anaerobic bacteria to be able to rapidly colonize the gastrointestinal tract of the animal, it will be understood that the bacteria must be viable and metabolically active after encapsulation in a porous capsule. For example, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% of the cells remain viable and metabolically active after encapsulation, than cells of the same type that were not encapsulated.

Before encapsulation, the strict obligate anaerobic bacteria are best grown to logarithmic (log) phase so that they are fully viable and metabolic active and harvested prior to encapsulation.

Methods of growing strict obligate anaerobes are known in the art. In an exemplary process, the strict obligate anaerobic bacteria is grown to log phase in a suitable anaerobic culturing media prior to encapsulation. The bacteria may be grown with or without shaking. Suitable examples of anaerobic culturing media includes, but are not limited to cooked meat broth, peptone-yeast extract glucose broth, MRS, thioglycollate broth, maltose media, and Yeast extract-Casitone-Fatty Acids (YCFA) medium supplemented with a starch or a sugar source (e.g., glucose). The anaerobic media may contain Rumen Fluid. In accordance with one example in which the bacteria utilizes lactic acid, the bacteria are grown in MRS broth. MRS broth may contain peptone, meat extract, yeast extract, glucose, tween-80, K₂HPO₄, sodium acetate, (NH₄)₂ citrate, MgSO₄-7H₂O and MnSO₄—H₂O. In accordance with another example in which the bacteria utilize starch, the bacteria are grown in maltose media with Rumen Fluid.

The strict obligate anaerobic bacteria are encapsulated with cellulose sulphate and pDADMAC e.g., according to the method of Dautzenberg et al. (1999b). Briefly, NaCS serves as polyanion and builds the capsule core. Poly[diallyldimethyl-ammonium chloride] solution as polycation provides a precipitation bath delivering the second reaction component for the polyelectrolyte complex formation at the surface of the cellulose sulphate capsule core, thus forming microcapsules by covering the NaCS core droplets with a solid membrane. Following culturing to logarithmic phase, a portion, for example 50 μ, 100 μl, or 200 μl of the bacterial culture is mixed with about 20 times (100 μl are mixed with 2 ml) of that volume of sodium cellulose sulphate solution containing 1.8% sodium cellulose sulphate (09-Sul-592, Fraunhofer Institute Golm, Germany) and 0.9% to 1% sodium chloride. Small amounts of that solution, for example droplets are then introduced into a bath of 1.3% 24 kDa (21-25 kDa average size) pDADMAC. This may be done with the use of a syringe and a needle, if no encapsulator is available or with the droplet generator system as described above. After a hardening time of 4 mins and several wash steps, the encapsulated cells are obtained from the bath and ready for use or storage.

Thus, disclosed herein is a method of producing a porous capsule comprising one or more strict obligate bacteria, the method comprising:
(i) culturing one or more strict obligate anaerobic bacteria in an anaerobic media;
(ii) encapsulating the bacteria in a capsule having a porous wall comprising surface pores with a molecular weight cut off between 50 and 200 kDa, wherein the porous wall comprises a complex formed from sodium cellulose sulphate and poly[dimethyldially-ammonium chloride]; and
(iii) performing one or more step which suspend growth of bacteria (e.g., in the log phase).

After encapsulation, the encapsulated strict obligate anaerobic bacteria may be further cultivated to enumerate the number of bacterial cells within each capsule e.g., such that the volume of each capsule is filled with the bacteria, which can be determined under a microscope. For example, each capsule may contain at least 1×10⁴ CFU of bacteria. For example, each capsule may contain at least 1×10³ CFU of bacteria. For example, each capsule may contain at least 0.5×10⁵ CFU of bacteria. For example, each capsule may contain at least 1×10⁵ CFU of bacteria. For example, each capsule may contain at least 0.4×10⁶ CFU of bacteria. In each of the foregoing example, the capsule may contain up to 1×10¹² CFU or more bacteria, depending on the size and volume of the capsule.

Once the capsules contain the desired number of bacterial cells, the encapsulated bacteria may be suspended in the log phase of growth within the capsules using any suitable method known in the art. For example, growth of the encapsulated bacteria may be suspended in the log phase using a method including, but not limited to, freeze-drying, spray-drying or extrusion. In one example, the capsules are freeze-dried.

It will be understood that protection is achieved if either a majority of cells is still viable or is still metabolically active or if more of the encapsulated cells remain viable when compared with unencapsulated cells which are treated under the same conditions. Metabolically active is understood as showing a reading on a UV-Vis spectrophotometer at 570 nm after incubation with resazurin which is reduced to fluorescent resorufin that is significantly different from the background or a negative control value.

It will be understood by the person skilled in the art that the cell density, as well as the concentrations of the NaCl may be varied. Furthermore, the formation of capsules does not need to be limited to the exact hardening time of 240 s. The NaCl solution may be replaced by a PBS solution or other buffer solutions.

The size of the capsules may be between about 200 μm and about 1,200 μm in diameter, if produced in an automated process involving an apparatus such as the encapsulator IE-50R and IEM-40 from EncapBioSystems, Switzerland, previously distributed by Inotech. In one example, the capsule size may be between 200-700 μm or between 200-500 μm.

Alternative production method may involve the use of Pasteur pipettes. When using pasteur pipettes for the manual production of capsules, the diameter of the microcapsules may be between about 3,000-5,000 μm.

It will be understood that the size of the capsule should otherwise not affect the survival times during processing and storage.

In one example, the capsules of the disclosure are between about 500 μm and 700 μm in diameter.

### Freeze-Drying

Following encapsulation of the strict obligate anaerobic bacteria in the porous capsule, the resulting encapsulated bacteria may be freeze-dried. Methods of freeze-drying are known in the art. An exemplary method are described in WO2015000972, the full contents on which is incorporated by reference herein.

In one example, the freeze-drying method may comprise at least two consecutive incubation steps. The encapsulated cells may be incubated in each of the incubation steps in an incubation solution containing cryoprotectant over a suitable period of time, wherein the concentration of cryoprotectant in the incubation solution is increased with each subsequent incubation step. This method may provide a protective effect on the (structural) integrity of capsules (the encapsulation material) both before and during the freeze-drying process.

In addition, the shelf-life of the capsules with the cells encapsulated therein is extended and the viability of the encapsulated cells increased. Without wishing to be bound by any one theory, it is believed that subjecting the encapsulated cells to the at least two consecutive incubation steps disclosed herein avoids capsules from “crumpling”.

The increase of the concentration of the cryoprotectant (it is noted here that the term “cryoprotectant” as used herein refers to both a single cryoproctant and a mixture/combination of two or more cryoprotectants) in the incubation solution during the consecutive incubation steps can be achieved in various ways. It is, for example, possible to add to a suspension of the encapsulated cells, for each incubation step a stock solution of the cryoprotectant. For example, if a cryoprotectant such as DMSO, formamide, N-methylacetamide (MA), or propanediol is used, a stock solution of the pure cryoprotectant 100% stock solution) might be used and in each incubation step a certain amount of the stock solution is added to the cell suspension to increase the concentration of the cryoprotectant. Alternatively, it is, for example, also possible to use the solution in which the encapsulated cells will be subjected to the freeze-drying (i.e., the freezing solution or cryopreservation medium) as a starting/stock solution to achieve an increasing in the concentration of the cryoprotectant. Using the final freezing solution for this purpose has the advantage that no extra stock solution has to be prepared for the consecutive incubation steps. This approach simplifies the handling of the incubation steps when a mixture of cryoprotectants are used in the incubation steps, for example, a mixture of skim milk powder with glycerol or a mixture of skim milk powder, glycerol and a carbohydrate such as sucrose or trehalose. In such a case, the prepared freezing solution (for example, 5% (w/v) skim milk and 1% (v/v) glycerol in water or an aqueous solution of 5% (w/v) skim milk, 1% (v/v) glycerol and 10% (w/v) of a carbohydrate such as sucrose or trehalose), is used to “serially dilute” in each incubation step the medium in which the encapsulated cells are stored. This “serial dilution” may, for example, be achieved as follows. Half the volume of the cell medium in which the encapsulated cells are present is removed from the respective vial, and the same volume of the freezing solution is added for the first incubation step. The encapsulated cells are then incubated for the desired period of time and then again 50% of the volume of the incubation mixture is removed and replaced by the same volume of freezing solution for the second incubation step. This procedure may be repeated as often as desired, thereby increasing the concentration of the cryoprotectant in each incubation step. The last incubation step may be carried out in the freezing solution.

The term “freeze-drying” (also known as lyophilisation, lyophilization, or cryodesiccation) is used in its regular meaning as the cooling of a liquid sample, resulting in the conversion of freeze-able solution into ice, crystallization of crystallisable solutes and the formation of an amorphous matrix comprising non-crystallizing solutes associated with unfrozen mixture, followed by evaporation (sublimation) of water from amorphous matrix. In this process the evaporation (sublimation) of the frozen water in the material is usually carried out under reducing the surrounding pressure to allow the frozen water in the material to sublimate directly from the solid phase to the gas phase. Freeze-drying typically includes the steps of pretreatment, freezing, primary drying and secondary drying.

The pretreatment includes any method of treating the desired product, i.e., encapsulated cells, prior to freeze-drying. The pretreatment may, for example, include washing the cells, formulation revision (i.e., addition of components to increase stability and/or improve processing), or decreasing the amount of a high vapor pressure solvent or increasing the surface area.

The freeze-drying step includes any method that is suitable for freeze-drying of the encapsulated cells. On a small scale, such as in a laboratory, freeze-drying may be done by placing the material in a freeze-drying flask and rotating the flask in a bath, also known as a shell freezer, which is cooled by, for example, mechanical refrigeration, by a mixture of dry ice with an alcohol such as methanol or ethanol, or by liquid nitrogen. It is of course also possible to use a commercially available freeze-dry apparatus such as Thermo Scientific® Modulyo Freeze-Dry System distributed by Thermo Fisher Scientific Inc. On a larger scale, freeze-drying is generally using a commercial, temperature controlled freeze-drying machine. When freeze-drying the encapsulated cells, the freezing is generally carried out rapidly, in order to avoid the formation of ice crystals. Usually, the freezing temperatures are between −50° C. and −80° C.

The next step is the primary drying. During the primary drying phase, the pressure is lowered (typically to the range of a few millibars), and sufficient heat is supplied to the material for the water to sublime. The amount of heat necessary can be calculated using the sublimating molecules' latent heat of sublimation. In this initial drying phase, about 95% of the water in the material is sublimated. This phase may be slow (can be several days in the industry), because, if too much heat is added, the material's structure could be altered.

Secondary drying can follow as the last step in freeze drying. The secondary drying phase aims to remove, if present, unfrozen water molecules, since the ice was removed in the primary drying phase. In this phase, the temperature is usually higher than in the primary drying phase, and can even be above 0° C., to break any physico-chemical interactions that have formed between the water molecules and the frozen material. Usually the pressure is also lowered in this stage to encourage desorption (typically in the range of microbars, or fractions of a pascal). After the freeze-drying process is complete, the vacuum is usually broken with an inert gas, such as nitrogen, before the freeze-dried encapsulated cells are packaged and/or stored for the further use.

As evident from the above, the present method belongs to the “pretreatment” as understood by the person skilled in the art and can be used together with any known methodology of freezing and drying material such as free or encapsulated cells as described herein.

Accordingly, since the at least two consecutive incubation steps can be carried out with any suitable following freeze-drying steps the method of freeze-drying, may comprise at least two consecutive incubation steps, wherein the encapsulated cells are incubated in each incubation step in an incubation solution containing cryoprotectant over a suitable period of time, wherein the concentration of cryoprotectant in the incubation solution is increased with each subsequent incubation step.

Any suitable number of the least two consecutive incubation steps can be carried as long as the number is sufficient to provide a desired effect on, for example, the viability of the encapsulated cells after the freeze-drying. Thus the method may comprise 3, 4, 5, 6, 7, 8, 9 or 10 incubation steps, wherein in each incubation step the concentration of the cryoprotectant is increased. The incubation in each of the incubation steps may be carried out over any suitable amount of time, for example, a time that is found to be able to achieve a desired long-term stability of the capsules and/or the viability of the encapsulated cells. A suitable incubation time as well as a suitable the number of incubation steps may be determined empirically, for example, by assessing the viability of the encapsulated cells after freeze-drying followed by (after a certain time period) re-hydrating of the cells.

The incubation time may be typically about several minutes to about several hours per incubation step. The incubation may be carried out either without agitation but also under agitation (such as, for instance, shaking or rolling) to improve the uptake of the cryoprotectant by the encapsulation material and the cells.

The same cryoprotectant or a mixture of the same cryoprotectant may be used in each incubation step. The cryoprotectant may be any compound that is able to provide protection during the freeze-drying against damage to the use encapsulation material or the encapsulated cell. Examples of suitable cryoprotectants include, but are not limited to, skim milk, glycerol, dimethylsulfoxide (DMSO), formamide, a mixture of formamide and DMSO, N-methylacetamide (MA), polyvinylpyrrolidone, propanediol (either 1,2-propanediol or 1,3-propanediol or a mixture of both), propylene glycol, serum albumin, a mixture of serum albumin with methanol, a carbohydrate and alginate. Examples of alginates that may be used as cryoprotectant include Satialgine® alginate or Algogel® alginate.

Examples of carbohydrates that may be used as cryoprotectant include, but are not limited to sucrose, glucose mixed with methanol, lactose, trehalose, raffmose, dextran, pectin, hydroxyethyl starch (HES), and cellulose sulphate.

It is also possible to use a mixture of two or more cryoprotectants in the incubation solution, for example, a mixture of skim milk with glycerol or a mixture of skim milk with a carbohydrate. In such embodiments, it is possible that the concentration of only one of the cryoprotectants is increased in the consecutive incubation steps while the concentration of the second (or any further) cryoprotectant is held constant during the course of the incubation. The concentration of the cryoprotectant may be held constant and the cryoprotectant may be chosen from sucrose, glucose mixed with methanol, lactose, trehalose, raffmose, or dextran. The concentration of skim milk may be increased in each of the at least two consecutive incubation steps while the concentration of the carbohydrate (for example, sucrose, glucose mixed with methanol, lactose, trehalose, raffinose, or dextran) may be held constant in the at least two consecutive incubation steps.

The encapsulated cells may be transferred, after the consecutive at least two incubation steps, into a suitable freeze drying medium without an intermediate washing step. By “washing step” is in particular meant a step in which the incubated cells are contacted with a washing buffer/medium that is devoid of the cryoprotectant.

Alternatively, the encapsulated bacterial cells are freeze-dried in the suitable freeze drying medium after the last incubation step. In these embodiments the freeze drying medium may also contain a cryoprotectant. In these embodiments the freeze drying medium contains the same cryoprotectant as the incubation solution.

Examples of suitable cryoprotectants that can be used in the freezing step (which can be carried out after the method of the present disclosure include, but are not limited to, skim milk, glycerol, dimethylsulfoxide (DMSO), formamide, a mixture of formamide and DMSO, N-methylacetamide (MA), serum albumin, a mixture of serum albumin with methanol, polyvinylpyrrolidone, propanediol, propylene glycol, a carbohydrate and alginate, to again mention only a few illustrative examples.

Examples of suitable carbohydrate based cryoprotectants include, but are not limited to sucrose, glucose mixed with methanol, lactose, trehalose, raffmose, dextran, pectin, hydroxyethyl starch (HES) and cellulose sulphate.

The freeze drying medium may be an aqueous solution that contains the one or more cryoprotectant which has been chosen for the freezing step.

In one example, the amount of freeze-dried encapsulated M. elsdenii produced by a method disclosed herein is about 0.1×10⁶ cfu/capsule to about 2×10⁶ cfu/capsule or about 0.1×10⁶ cfu/capsule to about 1.8×10⁶ cfu/capsule or about 0.1×10⁶ cfu/capsule to about 1.5×10⁶ cfu/capsule or about 0.1×10⁶ cfu/capsule to about 1.2×10⁶ cfu/capsule.

In one example, the amount of viable freeze-dried encapsulated M. elsdenii produced by a method disclosed herein is about 1×10⁶ cfu/ml to about 5×10⁶ cfu/ml or about 1×10⁶ cfu/ml to about 4×10⁶ cfu/ml or about 1×10⁶ cfu/ml to about 3×10⁶ cfu/ml. In one example, the amount of viable freeze-dried encapsulated M. elsdenii produced by a method disclosed herein is about 4×10⁶ cfu/ml.

In one example, the viability of the freeze-dried encapsulated M. elsdenii after rehydration after 1 hour in aerobic conditions is about 0.005×10⁶ cfu/capsule to about 0.02×10⁶ cfu/capsule or about 0.01×10⁶ cfu/capsule to about 0.02×10⁶ cfu/capsule or about 0.015×10⁶ cfu/capsule to about 0.02×10⁶ cfu/capsule.

In one example, the viability of the freeze-dried encapsulated M. elsdenii after rehydration after 1 hour in anaerobic conditions is about 0.02×10⁶ cfu/capsule to about 0.06×10⁶ cfu/capsule or about 0.03×10⁶ cfu/capsule to about 0.055×10⁶ cfu/capsule or about 0.04×10⁶ cfu/capsule to about 0.055×10⁶ cfu/capsule.

In one example, the viability of the freeze-dried encapsulated M. elsdenii after rehydration after 5 hours in anaerobic conditions is about 0.02×10⁶ cfu/capsule to about 0.05×10⁶ cfu/capsule or about 0.02×10⁶ cfu/capsule to about 0.045×10⁶ cfu/capsule or about 0.02×10⁶ cfu/capsule to about 0.04×10⁶ cfu/capsule.

The freeze-dried encapsulated bacteria may remain viable in the porous capsule when stored under anaerobic conditions for about 14 days to about 24 months at about −80° C., about −20° C., about 4° C., about 25° C., about 30° C., or combinations thereof. In one example, the freeze-dried encapsulated bacteria are viable in the porous capsule for at least about 14 days, at least about 1 month, at least about 4 months, at least about 6 months, at least about 8 months, at least about 10 months, at least about 12 months, at least about 15 months, at least about 18 months or at least about 24 months at ambient temperatures e.g., about 25° C.

In some examples, the freeze-dried encapsulated bacteria remain viable in the porous capsule for at least about 1 month at ambient temperature(s) when stored under anaerobic conditions. In some examples, the freeze-dried encapsulated bacteria remain viable in the porous capsule for at least about 2 month (e.g., at least about 3 month, or at least about 4 months, or at least about 5 months, or at least about 6 months, or at least about 7 months, or at least about 8 months, or at least about 9 months, or at least about 10 months, or at least about 11 months, or at least about 12 months or more) at ambient temperature(s) when stored under anaerobic conditions. In some examples, the freeze-dried encapsulated bacteria can remain viable in the porous capsule for about 24 month at ambient temperature(s) when stored under anaerobic conditions.

In one example, the porous capsule contains at least about 0.4×10⁵ CFU of the strict obligate anaerobic bacteria 1 month after encapsulation following storage in an anaerobic environment at ambient temperatures. In one example, the porous capsule contains at least about 0.4×10⁵ CFU of the strict obligate anaerobic bacteria 3 month after encapsulation following storage in an anaerobic environment at ambient temperatures. In another example, the porous capsule contains at least about 0.2×10⁵ CFU of the strict obligate anaerobic bacteria 8 month after encapsulation following storage in an anaerobic environment at ambient temperatures.

### Methods of Use

The inventors have shown surprisingly that the oral administration of porous capsules containing M. eldesdenii facilitates rapid adaptation of a ruminant animal to a diet having a relatively higher amount of fermentable carbohydrates, without apparent development of acidosis or symptoms thereof. This was demonstrated with the use of a 3 day ‘step up’ diet, whereas a step up diet of between 10 and 30 days is typically employed when transitioning livestock from a roughage based diet to a diet which is richer in fermentable carbohydrates to acclimate the animal to the increased availability of fermentable carbohydrate. Without wishing to be bound by any one theory, the inventors believe that the encapsulation of the M. eldesdenii within porous capsule preserves the viability of the bacteria and allows them to reach the rumen. Once in the rumen, the bacteria are able to exit the capsules through the porous capsule walls, where they rapidly colonize the rumen and utilize lactate produced during fermentation of carbohydrate sources. Metabolism of lactate produced through fermentation by M. eldesdenii assists in maintaining a physiologically stable pH in the rumen. At the same time, bacteria retained within the porous capsule receive nutrients from ruminal fluid (including lactate) and continue to propagate within the capsule, providing an ongoing source of bacteria for release into the rumen. Based on these finding, the inventors have developed and provide herein a number of methods and applications which involve the oral administration of the encapsulated strict obligate anaerobic bacteria e.g., including but not limited to M. eldesdenii and/or R. bromii, to animal (e.g., such as livestock), wherein the capsule is porous. The inventors also contemplate the use of this approach for delivery of other strict obligate anaerobic bacteria, including other lactic acid utilizing bacteria.

Accordingly, in one example, the present disclosure provides a method of orally administering to an animal a strict obligate anaerobic bacteria encapsulated within a porous capsule as described herein to increase a population of the bacteria in the gastrointestinal tract of the animal. For example, performance of the method of the disclosure may increase the population of the strict obligate anaerobic bacteria in the gastrointestinal tract by at least 10%, or at least 20%, or at least 30%, or at least 40%, or at least 50%, or at least 60%, or at least 70%, or at least 80%, or at least 90%, or least 100%, or least 150%, or least 200%, or least 300%, or at least 400% relative to the population prior to administration of the encapsulated bacteria and/or relative to an animal to which the encapsulated strict obligate anaerobic bacteria has not been in administered.

The present disclosure also provides a method of facilitating adaptation of an animal to a diet having a relatively higher amount of fermentable carbohydrates, said method comprising administering an encapsulated strict obligate anaerobic bacteria of the disclosure to the animal, wherein the bacteria is encapsulated in a porous capsule as described herein. Using the method of the disclosure, the timeframe in which it takes to adapt the animal to the diet having a relatively higher amount of fermentable carbohydrates may be within 5 days or less, or within 4 days or less, or within 3 days or less, or within 2 days or less or within 1 day of increasing the level of fermentable carbohydrate in the diet. In some examples, the animal may be fed a ‘step up’ diet during the period in which it takes the animal to adapt. In other examples, the animal is simply transitioned straight to the new diet having the relatively higher amount of fermentable carbohydrates.

As used herein, fermentable carbohydrates may include, but are not limited to, sources of starch e.g., wheat, triticale, sorghum, barley, maize, lupins and oats, and source of sugar e.g., molasses, and fibers.

In one example, a diet having a relatively higher amount of fermentable carbohydrates comprises a higher proportion of concentrates (e.g., sources of starch such as grains from wheat, triticale, sorghum, barley, maize, lupins and oats etc) relative to the proportion of roughage e.g., hay or silage.

The present disclosure also provides a method of reducing accumulation of organic acid, in particular lactic acid, in the gastrointestinal tract of an animal, comprising administering an encapsulated strict obligate anaerobic bacteria to the animal, wherein the bacteria is an lactic acid-utilizing bacteria encapsulated in a porous capsule as described herein.

The present disclosure also provides a method of maintaining a stable pH in the gastrointestinal tract of an animal by administering an encapsulated strict obligate anaerobic bacteria to the animal, wherein the bacteria is encapsulated in a porous capsule as described herein.

The present disclosure also provides a method of preventing or treating lactic acidosis, or one or more associated conditions or clinical symptoms thereof, in an animal, by administering an encapsulated strict obligate anaerobic bacteria to the gastrointestinal tract of the animal, wherein the bacteria is encapsulated in a porous capsule as described herein.

As referred to herein, “acidosis” or “lactic acidosis” refers to a metabolic disorder characterised by an accumulation of organic acids, especially lactic acid, in the GI tract (specifically the rumen and reticulum of ruminants, or the hind gut of hindgut fermenters) resulting in a decrease in pH of the rumen or hindgut. Lactic acidosis may be further categorised into sub-acute and acute acidosis. Sub-acute and acute acidosis are simply different degrees of the same problem. Acute rumen acidosis is more severe and physiological functions may be significantly impaired. The affected animal may present as being depressed and ataxic, off-feed, with dilated pupils and an elevated heart rate. Diarrhoea will be obvious and the animal may become recumbent and die within 2 to 5 days after the insult. Acute acidosis is typically characterised by a dramatic reduction in pH (below pH 5.0) within the rumen or hind-gut (depending on the gut anatomy), a large increase in lactic acid concentration and a large decrease in protozoa. Sub-acute acidosis, on the other hand, is typically characterised by a reduction in pH within the range of 5.6 to 5.2. The symptoms of sub-acute rumen acidosis differ from that of acute acidosis and can be difficult to recognise within a large group. Groups of animals with sub-acute acidosis will typically present some or all of the following signs: laminitis, intermittent diarrhoea, poor appetite or cyclical feed intake, high cull rates for poorly defined health problems, poor body condition in spite of adequate energy intake, abscesses without obvious causes and hemoptysis (coughing of blood) or epistaxis (bleeding from the nose). Most of these signs are secondary to acidosis and most of them do not appear until weeks or months after the initial acidosis events.

In almost all cases in livestock, acidosis is caused by a gross imbalance between the numbers of lactic acid-producing bacteria (LAB) and lactic acid-utilising bacteria (LUB) in the GI tract, typically brought on by a sudden increase in the proportion of readily fermentable carbohydrates in the animal's diet (e.g., an increase in grain and concentrate feed) and/or a lower proportion of roughage (e.g., hay, silage and other sources of structural carbohydrates). This in turn increases the production of lactic acid in the GI tract. Further, a reduction in structural carbohydrates necessary for stimulating mastication and digestion (e.g., rumination) reduce the animal's ability to buffer changes in acidity in the GI tract.

Methods of determining whether an animal is suffering from acidosis are known in the art and contemplated herein.

In one example, the method of the disclosure is used to treat or prevent sub-acute lactic acidosis. In another example, the method of the disclosure treat or prevent acute lactic acidosis. In yet another example, the method of the disclosure is used to prevent the progression of sub-acute acidosis to acute acidosis.

The one or more conditions associated with lactic acidosis may be selected from the group consisting of rumenitis, lactic acidosis induced laminitis, lactic acidosis induced bloat, polioencaphomelacia, colic, gastric ulcers and liver abscesses, as well as combination thereof. The one or more clinical symptoms of lactic acidosis may be selected from reduced feed intake, reduced feed-conversion efficiency, weight loss, lameness, diarrhea, dehydration, reduced physical performance, slow recovery from exercise, crib-biting, wind-sucking and weaving behaviour, as well as combinations thereof.

In one example, the animal is a ruminant and the administration of the encapsulated bacteria to the gastrointestinal tract of the animal increases pH of the rumen and/or maintains pH of the rumen above 5.5. For example, the pH of the rumen may be maintained between about 6.2 and 7.0.

In another example, the animal is a hindgut fermenter and administration of the encapsulated bacteria to the gastrointestinal tract of the animal increases pH of the hindgut and/or maintains pH of the hindgut above about 5.5. For example, the pH of the rumen may be maintained between about 6.5 and 7.0.

In one example, the present disclosure provides a method of stabilising a fermentative process of digestion and/or optimising microbial populations and function within the gastrointestinal tract of an animal, comprising administering an encapsulated strict obligate anaerobic bacteria to the animal, wherein the bacteria is encapsulated in a porous capsule as described herein.

In one example, the present disclosure provides a method of improving feed conversion efficiency or feed efficiency in a livestock animal by administering to the animal an encapsulated strict obligate anaerobic bacteria as described herein and/or a composition comprising same as described herein. Feed conversion (or feed conversion ratio or feed conversion rate) is a measure of the efficiency with which the bodies of livestock convert animal feed into the desired output. For example, for dairy cows the desired output is milk, whereas in animals raised for meat, such as beef cattle, the output is meat, or the body mass of the animal. Feed conversion is the mass of the input divided by the output. In contrast, feed efficiency is the output divided by the input (i.e. the inverse of feed conversion ratio).

In one example, the present disclosure provides a method of improving starch utilisation in the gastrointestinal tract of an animal, comprising administering an encapsulated strict obligate anaerobic bacteria to the animal, wherein the bacteria is encapsulated in a porous capsule as described herein. Methods of determining starch utilisation are known in the art and contemplated herein. In some example, the method may improve starch utilisation by at least 10%, or at least 20%, or at least 30%, or at least 40%, or at least 50%, or at least 60%, or at least 70%, or at least 80%, or at least 90%, or least 100% relative to the animal's utilisation of starch prior to administration of the encapsulated strict obligate anaerobic bacteria and/or relative to an animal to which the encapsulated strict obligate anaerobic bacteria has not been in administered.

In another example, the present disclosure provides a method of inducing satiety and/or controlling the food intake in an animal, comprising administering an encapsulated strict obligate anaerobic bacteria to the animal, wherein the bacteria is encapsulated in a porous capsule as described herein. As used herein, the term “satiety” shall be understood to refers to satisfaction of the need for nutrition and the extinguishment of the sensation of hunger, which is often described as “feeling full”. The satiety response refers to behavioural characteristics observed to be consistent with having consumed a sufficient amount of food, such as an abrupt or a tapered down cessation of eating. However, the biological mechanisms which lead to the satiety response are often triggered in a gradual or delayed manner, such that they are usually out of phase with the amount of food taken in by the animal prior to cessation, which results in the animal consuming more nutritional content than is appropriate or most efficient.

The method of the disclosure may be performed on any animal. However, exemplary animals for which the methods of the disclosure may be particularly useful include livestock species (e.g. cattle, sheep, horses, pigs, donkeys, poultry), companion animals (e.g. dogs, cats), performance animals (e.g. racehorses, camels, greyhounds) and captive wild animals. In one example, the “animal” is a ruminant. Exemplary ruminants include cattle, sheep, goats, buffalo, deer or camelids. In another example, the animal may be a hind gut fermenter. An exemplary hindgut fermenter is a horse. In another example, the animal may be an avian species, such as poultry. Whilst it is contemplated that the methods of the present disclosure may be particularly useful in non-human animals, it is also contemplated that the methods may be performed on humans. Accordingly, in one example, the animal is a human.

The encapsulated strict obligate anaerobic bacteria and/or compositions comprising same may be administered to an animal by any administration route determined to be suitable by a person skilled in the art. For example, the porous capsule in which the strict obligate anaerobic bacteria are encapsulated may be administered to the animal orally (e.g., as an ingestible liquid or solid, an oral drench, a feed additive, a food, a composition, or a capsule), intranasally or parenterally. In a particularly preferred example, the capsule in which the strict obligate anaerobic bacteria are encapsulated and/or compositions comprising same is administered to the animal orally e.g., as a drench or feed supplement.

The appropriate dosage to be administered to the animal will be dependent on a range of factors, including, but not limited to, the species of animal, anatomy of the digestive system (e.g., four chamber or single chamber stomach), the size of the animal, the composition of the animal's diet (existing and future), whether the animal is lactating, whether the animal is pregnant and the outcome to be achieved. The appropriate dosage of bacteria (e.g., CFUs per strain) to be delivered to an animal may be determined by a person skilled in the art taking into account one or more of the above factors.

In one example, the methods of the disclosure comprises administering one or more capsules amounting to a dosage between about 10² CFU to about 10¹⁴ CFU, or about 10³ CFU to about 10¹³ CFU, or about 10⁴ CFU to about 10¹³ CFU, or about 10⁵ CFU to about 10¹³ CFU, or about 10⁶ CFU to about 10¹³ CFU, or about 10⁶ CFU to about 10¹² CFU, or about 10⁷ CFU to about 10¹¹ CFU, or about 10⁸ CFU to about 10¹⁰ CFU, or about 10⁹ CFU to about 10¹⁰ CFU. For example, each dosage of encapsulated strict obligate anaerobic bacteria may comprise about 5×10⁷ CFU or about 6×10⁸ CFU, or about 10⁹ CFU, or about 10¹⁰ CFU of the bacteria.

In one embodiment, the encapsulated strict obligate anaerobic bacteria according to the methods and uses of the present disclosure is administered once or more daily, weekly, fortnightly, monthly, or bi-monthly, wherein a daily, weekly, fortnightly, monthly, or bi-monthly dosage comprises an amount of the strict obligate anaerobic bacteria as described above. In one embodiment, the capsule in which the strict obligate anaerobic bacteria is encapsulated is administered weekly, wherein each dosage comprises an amount of the strict obligate anaerobic bacteria as described above. In one embodiment, the capsule in which the strict obligate anaerobic bacteria is encapsulated as described herein is administered monthly, wherein each dosage comprises an amount of the strict obligate anaerobic bacteria as described above.

### Compositions

As described herein, the porous capsules in which the strict obligate anaerobic bacteria are encapsulated maybe provided in the form of a composition.

The composition may be provided in single dosage form or in multi-dosage form.

In some examples, the composition may be formulated for oral administration e.g., as a feed additive, bolus or drench.

Accordingly, the composition may further comprise one or more physiologically acceptable excipients, carriers or additives suitable for ingestion by an animal. Physiologically acceptable excipients, carriers or additives suitable for ingestion by an animal are known in the art and described herein. Such carriers can, for example, allow the encapsulated strict obligate anaerobic bacteria or feed additive of the disclosure to be formulated as tablets, pills, dragées, capsules, liquids, gels, syrups, slurries, suspensions and the like. The choice of carrier will be dependent on the form of the composition and intended method of administration (e.g., as a drench, as a top dress feed additive, as a capsule). In some examples, the composition may be a tablet, pill, caplet, or capsule. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, and polyvinylpyrrolidone (PVP). If desired, disintegrating agents can be added, such as, but not limited to, the cross-linked polyvinylpyrrolidone, agar, or alginic acid or a salt thereof such as sodium alginate. Compositions that can be used orally include, but are not limited to, capsules made of gelatin, as well as soft, sealed capsules made of gelatin and a plasticizer, such as glycerol or sorbitol. In some examples, the composition may be formulated in a buffer. It will be understood by a person skilled in the art that by suitable buffer may be used. Examples of suitable buffers include, but are not limited to phosphate, calcium carbonate, bicarbonate, phosphate citrate and histidine. In other examples, the composition may be formulated with a carrier having a low oxygen diffusion rate e.g., such as ingestible oils. The composition may further comprise an antioxidant.

In some example, the composition is provided in a wet form (e.g., a gel or liquid). In other examples, the composition may be provide in a dry or solid form (e.g., a flowable powder), granule (i.e., a granulate), particle (i.e., particulate), pellet, cake, water soluble concentrate, paste, bolus, tablet, dust, a component thereof, or combinations thereof.

In some examples, the composition may comprise a preservative or a stabilizer. Furthermore, depending on the method of manufacture, the composition may comprise one or more cryoprotectants as described herein.

In accordance with an example in which the composition is an animal feed supplement, the composition may be prepared by, or shipped to, an animal feed manufacturer. The composition may then be formulated into a nutritional supplement for specific animals (e.g., specific livestock species) by the addition of further ingredients including a bulking agent (for example, canola meal, wheat and/or rice hulls) and optionally additional minerals and ingredients, such as, for example copper, acid buffer, magnesium oxide, potassium chloride, sulphur, salt, lime, and/or vegetable oil.

Alternatively, a composition described herein may be formulated as an animal feed, i.e. a full feed ration, comprising further ingredients such as wheat, barley, corn, lupins, chickpeas, hay and/or molasses. As would be understood in the art, animal feeds will typically be nutritionally complete.

In some examples, the composition of the disclosure is stable when stored under anaerobic conditions at ambient room temperature (e.g., 20° C. and 25° C.) for at least one month, or at least 2 months, or at least 3 months, or at least 4 months, or at least 5 months, or at least 6 months or more. In some examples, the composition of the disclosure is stable at ambient temperatures for 12 months when stored under anaerobic conditions. As used herein, the term “stable” shall be understood to mean that the composition will lose less than 1.5 log CFU of the strict obligate anaerobic bacteria, and preferably less than 1 log CFU of the strict obligate anaerobic bacteria after the designated period of time.

In one particular example, the disclosure provides a composition in which the capsules comprising the strict obligate anaerobic bacteria are resuspended in an oil carrier and dispensed via a capsule.

In some examples, the composition of the disclosure is packaged under anaerobic conditions. Accordingly, the present disclosure provides a porous capsule or composition as described herein packaged in a container under anaerobic conditions. The container may contain a single dose or multiple doses of the porous capsule or composition comprising same as described herein.

### EXAMPLES

### Example 1. Growing of Anaerobic Cultures

### Preparation of MRS Media

5.1 g MRS broth, 0.1 g Polysorbate 80, 0.2 ml Resazurin were added to 99 ml (+10 ml excess) MilliQ H2O in 250 ml serum bottle. The MRS media was boiled under constant flow of gas mix (95% CO2/5% H2) for 20 mins. After boiling, 0.05 g Cysteine-HCl was added to the media and brought to autoclave at 105° C. for 45 mins.

### Preparation of Lactic Acid Media

VFA solution (pH 7.5), Salt Solution A and Salt Solution B were prepared according to Tables 1-3 below. Lactic acid media was prepared according to Table 4. The lactic acid media was boiled under constant flow of 95%/CO₂/5% H₂ until the solution turned straw coloured. Once cooled, VFA solution and Cysteine-HCl were added and the media was mixed well before use. Media vessels were prepared by gassing with 95%/CO₂/5% H₂.

TABLE 1
VFA solution

| Component | Volume per L |
|---|---|
| Acetic acid | 170 ml |
| Propionic acid | 60 ml |
| Butyric acid | 40 ml |
| Isobutyric acid | 10 ml |
| n-Valeric acid | 10 ml |
| Isovaleric acid | 10 ml |
| D-L-α methyl butyric acid | 10 ml |

TABLE 2
Salt solution A

| Component | Mass per L |
|---|---|
| KH₂PO₄ | 3.0 g |
| NaCl | 6.0 g |
| (NH4)₂SO₄ | 3.0 g |
| CaCl₂ | 0.3 g |
| MgSO₄ | 0.3 g |

TABLE 3
Salt solution B

| Component | Mass per L |
|---|---|
| K₂HPO₄ | 3.0 g |

TABLE 4
Lactic acid media

| | Mass or volume | |
|---|---|---|
| Component | per L of media | |
| Peptone | 1.0 | g |
| Yeast extract | 1.0 | g |
| NaHCO₃ | 5.0 | g |
| Lactic acid (85%) | 5 | mL |
| Distilled H₂O | 330 | mL+ |
| Salt Solution A | 165 | mL |
| Salt Solution B | 165 | mL |
| Rumen fluid | 33.0 | mL |
| Resazurin | 10.0 | mL |
| Add after boiling | | |
| VFA solution | 10 | mL |
| Cysteine-HCl | 0.20 | g |

### Growth of Cultures

Megasphaera elsdenii w and Ruminicoccus bromii were obtained from the Department of Agriculture and Fisheries in QLD.

M. elsdenii was inoculated into anaerobic lactic acid media and grown at 39° C. without shaking. R. bromii was inoculated into Maltose media with Rumen Fluid. The growth of the bacterial cultures were measured every 30 min by measuring the OD₆₀₀ₙₘ to determine log phase. Once the M. elsdenii reached an OD₆₀₀ₙₘ of 0.9 and the R. bromii reached an OD₆₀₀ₙₘ of 0.5, the bacteria were encapsulated.

### Example 2. Encapsulation of Anaerobic Bacterial Cultures

Preparation of Anaerobic SCS and pDADMAC

The SCS was filtered through a 0.22 μm filter through the gas line and gas was bubbled into SCS solution for 30 min. The presence of oxygen may be checked by adding a drop or two of SCS into the dilution solution/media as oxygen will turn the SCS pink.

pDADMAC/PBS was made by adding 10 mL of 10× PDADMAC solution, 90 mL dH₂O and 200 μl resazurin, boiling and running the solution under constant flow of 95%/CO₂/5% H₂ prior to autoclaving at 105° C. at 45 min. PBS was also made by bubbling the solution through 95%/CO₂/5% H₂. Plastics to be used were also flushed three to four times with 95%/CO₂/5% H₂ in an air lock chamber and placed in the anaerobic chamber for a week to remove oxygen from the plastics.

The solution was purged with 95%/CO₂/5% H₂ for two cycles (10 purges/cycle).

Encapsulation of M. elsdenii

1 ml of log phase bacterial culture (OD₆₀₀ₙₘ=0.9) was mixed with 1 ml of SCS and dropped into 100 ml of pDADMAC bath. The bacteria was hardened in the pDADMAC for 5 min then washed in PBS for 10 min, then 5 min followed by three quick washed) and finally followed by three washes in MRS media.

The resulting encapsulated bacteria were cultured in 100 ml of MRS media in 200 ml serum bottle at 39° C. without shaking. At 19 hours of culture, the capsules were 70% filled with bacteria. At 24 hours of culture, about 70% of the capsules had bacteria grown to fill up the whole capsule and about 30% of the capsules had bacteria filled up to 80% of the capsule. Capsules were frozen in 23 vials of ˜1000 caps in 1 mL per Wheaton 2R vial and stored at −80° C.

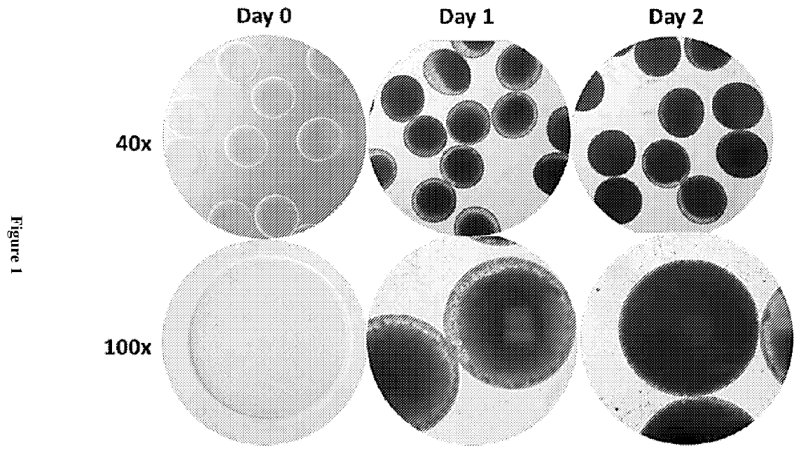

At 43 hours of culture, the capsules had bacteria which filled up the capsule resulting in whitish full capsules. Capsules were frozen in 11 vials of ˜1000 caps in 1 mL per Wheaton 2R vial and stored at −80° C. FIG. 1 shows encapsulated M. elsdenii.

Encapsulation of R. bromii

1 ml of log phase bacterial culture (OD₆₀₀ₙₘ=0.5) was mixed with 1 ml of SCS and dropped into 100 ml of pDADMAC bath. The bacteria was hardened in the pDADMAC for 5 min then washed in PBS (10 min, then 5 min then three washes) followed by three washes in maltose media without Rumen Fluid.

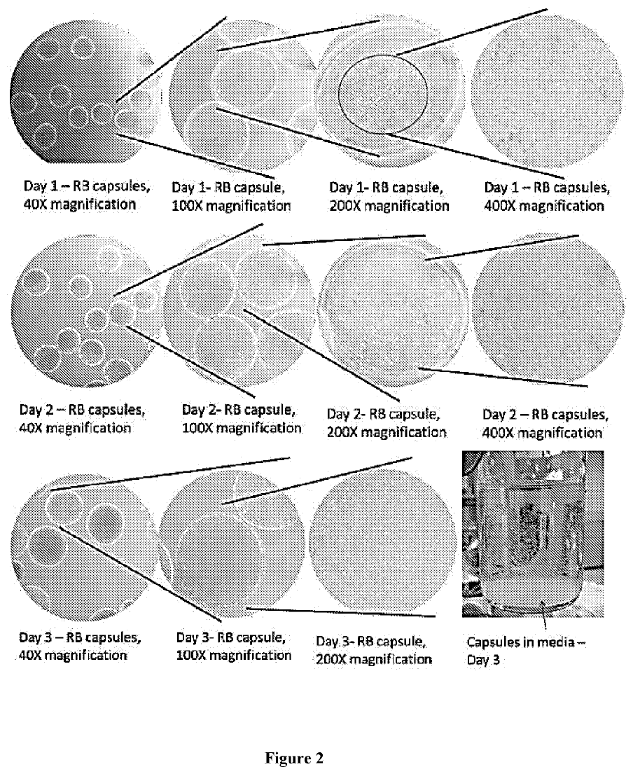

The resulting encapsulated bacteria were cultured in 250 ml of maltose media without Rumen Fluid in 200 ml serum bottle at 39° C. without shaking. At Day 1 post encapsulation, filamentous bacteria were seen inside the capsules. Capsules were 30% full with bacterial filaments. At Day 2 post encapsulation, capsules were observed to be 60% full with long filaments. At Day 3 post encapsulation, most capsules were 80% full with long filaments. Capsules were frozen at Day 3 post encapsulation in ten vials of ˜1000 caps in 1 mL per Wheaton 2R vial and stored at −80° C. FIG. 2 shows encapsulated R. bromii.

### Example 3. Freeze-Drying of Encapsulated Anaerobic Bacterial Cultures

Freeze drying was performed according to WO2015/000972 (AU2014286177) with the following modification.

### Anaerobic Freezing Media

Freezing media was prepared by the addition of 5% skim milk, 10% Trehalose and 1% glycerol in 100 ml dH₂O and autoclaved at 105° C. for 45 mins. After autoclaving, the freezing media was bubbled through a 0.22 μm filter under constant flow of gas mix of 95% CO₂/5% H₂ for 30 min.

### Example 4. Survival of Freeze-Dried Encapsulated Anaerobic Bacteria

M. elsdenii

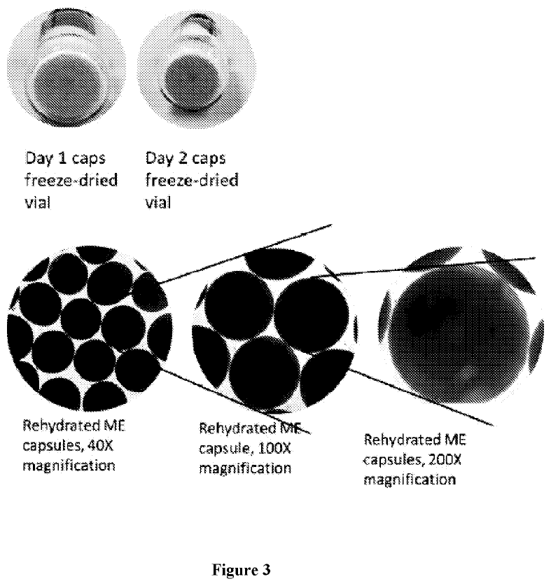

The freeze dried capsules containing M. elsdenii were rehydrated (FIG. 3) in lactic acid media and decapsulated according to WO/2015/171077 under anaerobic conditions. The rehydrated and decapsulated bacteria were plated on lactic acid media agar places and analysed for CFU to determine the number of bacteria per capsule.

R. bromii

Five vials of Day 3 R. bromii capsules were freeze-dried under standard freeze drying conditions. All five vials were well-dried. One vial was rehydrated in Maltose media with Rumen Fluid, decapsulated & underwent Prestoblue Assay and Growth curve monitoring.

### Metabolic Activity by Prestoblue Assay

At Day 0, bacteria stock was inoculated into Maltose media with Rumen Fluid and R. bromii capsules were rehydrated in Maltose media with Rumen Fluid. At Day 1, the controls, consisting of 5 μL, 50 μL and 200 μL of OD₆₀₀ₙₘ reading of 0.5 free bacteria, were placed in 96-well plate in quadruplicates.

At Day 1, ten rehydrated RB capsules were placed into 96-well plate in quadruplicates.

Prestoblue (bubbled with CO₂/H₂ to make anaerobic) was added to the samples in the plate and incubated and fluoscence was then measured in the Tecan machine as per manufacturer's instructions.

50, 100, 500 freeze-dried rehydrated capsules were decapsulated and put into Maltose media to monitor growth. 0.1 ml, 0.2 ml, 0.5 ml RB free bacteria were inoculated into Maltose media as a free bacteria control.

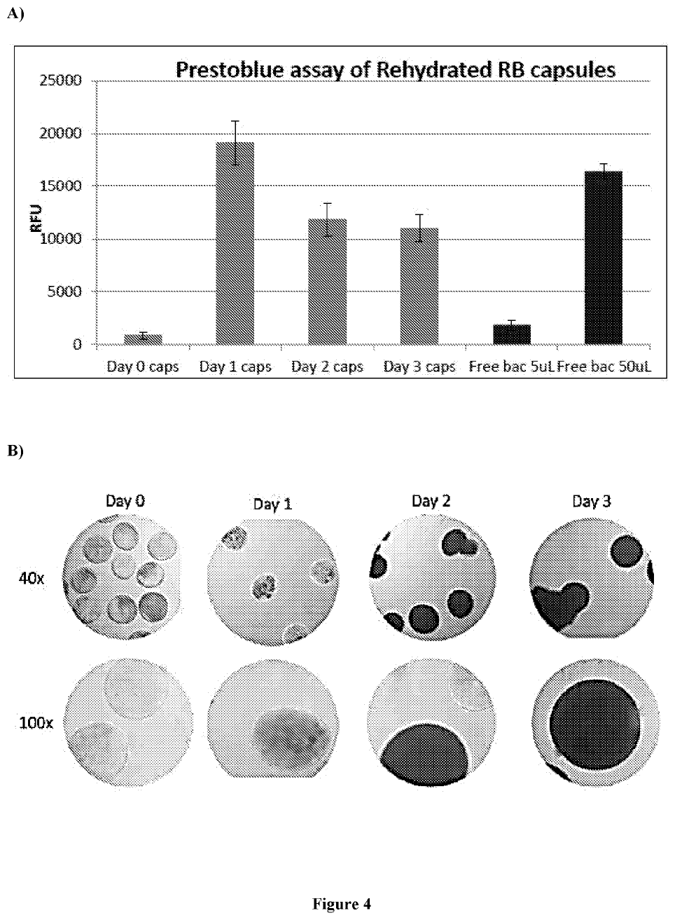

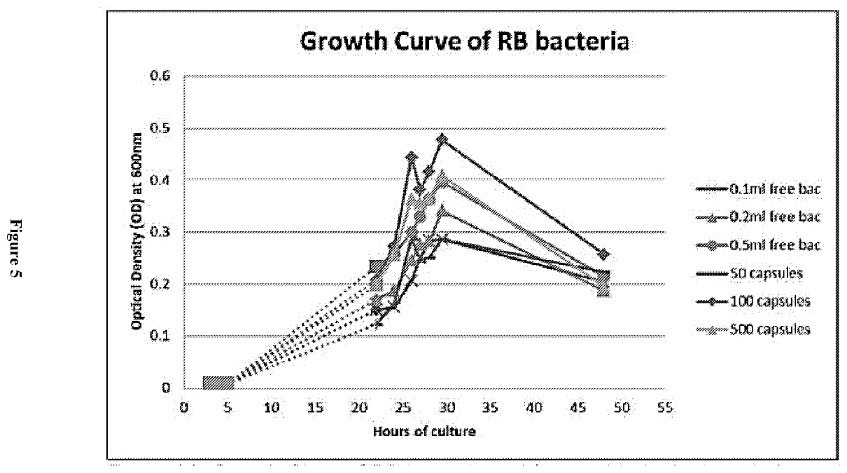

The OD₆₀₀ₙₘ of both free bacteria and decapsulated bacteria was read at the following timepoints: 22 hours, 24 hours, 26 hours, 27 hours, 28 hours, 29.5 hours and 48 hours showing that the bacteria are growing and viable (FIGS. 4 and 5).

### Example 5. Comparison of Viability of Encapsulated M. elsdenii and Free M. elsdenii

The viability of encapsulated M. elsdenii before and after freeze-drying was measured and compared to the viability of free M. elsdenii before and after freeze-drying.

TABLE 5
Viability of encapsulated M. elsdenii

| | Before | After |
|---|---|---|
| | freeze-drying | freeze-drying |
| | (×10⁶ | (×10⁶ |
| | cfu/cap) | cfu/cap) |
| Test batch | 5 | 1.18 |
| Batch 1 | 2.7 | 0.5 |
| Batch 2 | 1 | 0.18 |
| Batch 3 | 1.2 | 0.2 |

TABLE 6
Viability of free M. elsdenii

| | Before | After |
|---|---|---|
| | freeze-drying | freeze-drying |
| | (×10⁶ | (×10⁶ |
| | cfu/ml) | cfu/ml) |
| Free bacteria | 52 | 4 |

As shown in Tables 5 and 6, encapsulated M. elsdenii showed better survival (5-fold loss) compared to free M. elsdenii (13-fold loss).

### Example 6. Stability of Freeze-Dried Encapsulated Anaerobic Bacteria

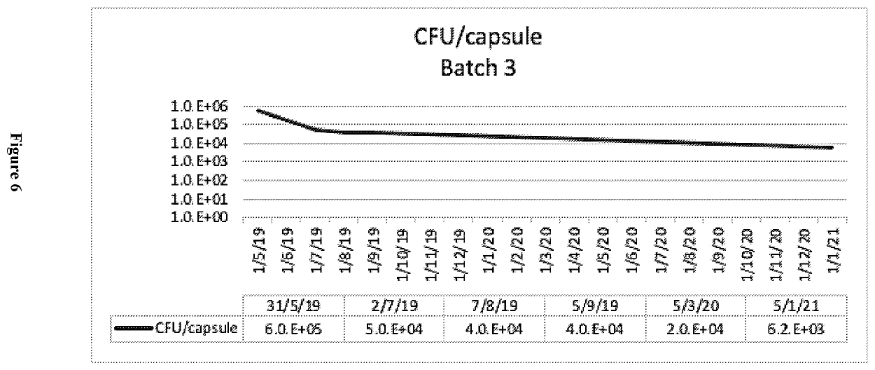

The shelf-life of the initial batches of freeze-dried encapsulated M. elsdenii was measured and found to be stable for 20 months at ambient temperature with 1-log loss in the first month of ambient temperature storage (FIG. 6). In this regard, one particular batch of encapsulated M. elsdenii was tested at 8 months post encapsulation following storage under anaerobic conditions at ambient temperature and shown to contain 2×10⁴ CFU/cap. This same batch was shipped from Singapore to Australia at ambient temperatures, where it was stored in a warm office for several months, after which time it was shipped back to Singapore under ambient temperatures for further assessment. At 20 months post encapsulation and following storage under anaerobic conditions at ambient temperature (including international transport), this batch was shown to contain 6,200 CFU/cap. Visual inspection of vials containing the encapsulated M. elsdenii also showed little change in colour of the capsule, which is indicative that the capsules contain viable bacteria beyond 20 months.

### Example 7. Oxygen Protection Data

The viability of encapsulated M. elsdenii after rehydration in aerobic and anaerobic conditions was measured and compared to the viability of free M. elsdenii after rehydration in aerobic and anaerobic conditions.

TABLE 7
Viability of rehydrated encapsulated M. elsdenii

| | | After | After |
|---|---|---|---|
| | | rehydration | rehydration |
| | Before | in aerobic | in anaerobic |
| | freeze-drying | conditions | conditions |
| | (cfu/cap) | (cfu/cap) | (cfu/cap) |
| After 1 h | 1 × 10⁶ | 0.015 × 10⁶ | 0.055 × 10⁶ |
| After 5 h | | 0 | 0.036 × 10⁶ |

TABLE 8
Viability of rehydrated free M. elsdenii

| Before freeze-drying (×10⁶ cfu/ml) | 52 |
|---|---|
| After rehydration in aerobic conditions | 0 |
| (×10⁶ cfu/ml) | |
| After rehydration in anaerobic conditions | 4 |
| (×10⁶ cfu/ml) | |

As is evidence from Tables 7 and 8, encapsulation increased the survival of M. elsdenii after rehydration in aerobic conditions.

### Example 8. Population Growth of M. elsdenii in Feed Lot Ration

The viability of M. elsdenii in freeze dried vials was tested in an in vitro batch culture experiment.

### Media

The Modified RF+ Medium was prepared in four batches of 1 L according to Table 9 and aliquoted into 450 mL volumes into gassed Wheaton bottles. The same batches of Salts A & B, Rumen fluid base and VFA solution were used for all four batches of media.

TABLE 9
Modified RF⁺ Medium

| | Mass or volume | |
|---|---|---|
| Component | per L of media | |
| NaHCO₃ | 5.0 | g |
| Distilled H₂O | 330 | mL |
| Salt Solution A | 165 | mL |
| Salt Solution B | 165 | mL |
| Lactic acid (85%) | 2.5 | mL |
| Rumen fluid base | 330 | mL |
| Resazurin | | |
| Add after boiling | | |
| VFA solution | 10 | mL |
| Cysteine-HCl | 0.20 | g |

### Genomic DNA Extraction

Genomic DNA (gDNA) was extracted from 1.0 mL cell pellets using a modification of the RBB+C method of Yu and Forster, 2005 with 300 μL dH₂O added to the eluted gDNA to a final volume of 500 μL. The quality and quantity of the extracted gDNA was determined by 1% agarose gel electrophoresis in Tris Acetate EDTA (TAE) buffer along with a 5.0 μL aliquot of GeneRuler 1 Kb DNA ladder (1:5) (Thermo Fisher Scientific) and the DNA was visualised using GelRed® stain (Biotium, USA).

qPCR Assay

The numbers of M. elsdenii cells present in collected samples were determined following the quantitative PCR assay method of Ouwerkerk et al., 2002. In brief, to prepare the quantitative standards, M. elsdenii YE34 was grown in broth culture at 39° C. overnight. The number of M. elsdenii YE34 cells was determined using a Petroff-Hauser Bacteria Counter (Arthur H. Thomas Company, Philadelphia, PA, USA), as per the manufacturer's instructions, at a magnification of 400× with an Olympus BH-2 microscope. The gDNA was extracted from a known number of bacterial cells and used in a dilution series to prepare six standards ranging in cell numbers from 1×10⁹ cells/mL down to 1×10⁴ cells/mL.

The primer and probe sequences are shown in Table 10. The probe was labelled at the 5′ end with the fluorescent reporter dye 6-carboxyfluorescein (6FAM) and at the 3′ end with the quencher dye 6-carboxy tetramethylrhodamine (TAMRA).

TABLE 10
DNA sequences of the M. elsdenii qPCR
assay primers and probes

| Name | Sequence |
|---|---|
| MelsF (primer) | 5′-GACCGAAACTGCGATGCTAGA-3′ |
| MelsR (primer) | 5′-GACCGAAACTGCGATGCTAGA-3′ |
| MelsP (probe) | 5′-TCCAGAAAGCCGCTTTCGCCACT-3′ |

The assay volume for the quantitative PCR was 25 μL and the components added to 0.1 mL tube are detailed in Table 11. Each quantitative PCR run included the standards run in triplicate, a no template control (NTC) run in triplicate and samples run in triplicate.

TABLE 11
qPCR assay

| Reagent | Volume (μL) |
|---|---|
| Precision Master Mix (2X) | 12.5 |
| MelsF | 0.5 |
| MelsR | 0.5 |
| MelsP | 0.25 |
| dH₂O | 6.25 |
| Template gDNA | 5 |

The quantitative PCR was performed on a Corbett Rotor-Gene 6000 with a run cycle of 94° C. for 1 min followed by 40 cycles of 94° C. for 10 s and 60° C. for 30 s. The resulting data was initially analysed using the Rotor-Gene Q Software V 2.3.4.3 and exported to a Microsoft Excel spreadsheet for further analysis.

### Rumen Fluid Inoculum Collection

### Collection Method

The two rumen fistulated steers (#1989 and #1990) that were available for the experiment are held at the DAF Dairy located on the University of Queensland's Gatton Campus, approximately a 1 hour 20 minute drive from the Rumen Ecology and Nutrition Unit (RENU) laboratory. Tests were undertaken to:
1. ensure the rumen fluid collected was maintained anaerobically at a constant temperature close to rumen temperature during transfer back to laboratory; and
2. measure the background levels of M. elsdenii present the rumen fluid.

Rumen fluid was collected from the steers into two 500 mL stainless steel thermos flasks which were preheated with hot water. The temperature was measured upon arrival at the RENU labs and found to be at 35° C. Aliquots of rumen fluid (1.0 mL) were taken from both thermos flasks, placed into 1.5 mL microcentrifuge tubes, centrifuged at 17,000×g for 10 min, the resulting supernatant removed and the remaining cell pellet stored frozen at −20° C. for future gDNA extraction and use as template in the M. elsdenii quantitative PCR assay.

### Batch Culture Experiment

The ProAgni feedlot ration (ProAgni ProTect 5%, wheat 20%, lupins 5%, Urea 0.3%, corn 54.7%, Rhodes grass hay 20%) was pre-weighed into 3.0 g amounts in 20 mL white capped vials and taken into the Anaerobic Chamber (Coy, Michigan USA) four days before the experiment and allowed to equilibrate to anaerobic conditions.

The 450 mL Wheaton bottles of modified RF+ media were placed at 39° C. on the morning of the experiment. Rumen fluid was collected from steer #1990 at UQ Gatton, placed into two 500 mL stainless steel thermos flasks, transported back to the RENU laboratories and taken into the anaerobic chamber. The 450 mL Wheaton bottles of modified RF+ media were taken into the anaerobic chamber and a 3.0 g vial of Proagni feedlot ration and 50 mL of well mixed rumen fluid was added to each bottle.

For each of the 3 treatment replicates a vial of freeze dried encapsulated M. elsdenii was mixed with 1.0 mL of modified RF+ media and transferred to the Wheaton bottle. The vial was rinsed another 3 times using fluid drawn up from the Wheaton bottle and the vial inspected visually to ensure no M. elsdenii beads remained in the vial. For the control groups a 1.0 mL aliquot of modified RF+ media was added to each Wheaton bottle.

After setup, at time 0 h, all bottles were mixed well and three 1.0 mL aliquots of fluid were taken (time 0 h) and placed into 1.5 mL microcentrifuge tubes, centrifuged at 17,000×g for 10 min, the resulting supernatant removed and the remaining cell pellet stored frozen at −20° C. for future gDNA extraction (see above) and used as template in the M. elsdenii quantitative PCR assay (see above). A 2.0 mL sample was taken and the pH measured using an Oakton pH Spear (Thermofisher Scientific) from 0 h to 6 h and then a portable Oakton pH meter was used for the 19, 24 and 48 h measurements.

A drop of the culture fluid was placed on a slide, viewed under 400× magnification on a Nikon eclipse 80i microscope and a representative image of a field of view taken. The bottles were incubated at 39° C. on an angle with shaking at 120 rpm and sampled hourly as described previously until 6 hours then again at 19 h, 24 h and 48 h. Three 1.0 mL samples were also taken from an unused bottle of modified RF+ media and from the rumen fluid as processed as described previously.

### Results

The quantitative PCR assay (Ouwerkerk et al., 2002) was tested against using genomic DNA extracted from pure culture from a panel of common rumen bacterial isolates and found late cycle amplification occurred (cycles 37 to 40) with a couple of unrelated rumen bacteria (Bacteriodes fragilis and Ruminococcus flavifaciens) (Ouwerkerk et al., 2002). These bacteria are not closely related to M. elsdenii and do not have sequences in common with the primers or probe. Thus, it was assumed that this product was an inefficiently produced non-specific product and an arbitrary cut-off was assigned to the assay of 10⁴ cells/mL.

Pre-experiment testing of rumen fluid from the fistulated steers detected M. elsdenii but the levels were well below the quantitative PCR cut-off at 2.6×10² cells/mL and 7.74×10² cells/mL for steer #1989 steer #1990 respectively. To further reduce the population of M. elsdenii within their rumens, the steers were fed a low quality wheaten hay for seven days prior to the experiment.

The vials of freeze dried and encapsulated M. elsdenii were reconstituted in the anaerobic chamber by the addition of 1.0 mL of modified RF+ media to each vial. Each reconstituted vial was added to a Wheaton bottle containing modified RF+ media. Immediately after addition of the reconstituted freeze dried and encapsulated M. elsdenii, beads could seen floating on the surface of the media (not shown). However, after an hour of incubation at 39° C., with shaking, the encapsulated beads were no longer visible (not shown) in any of the Wheaton treatment bottles.

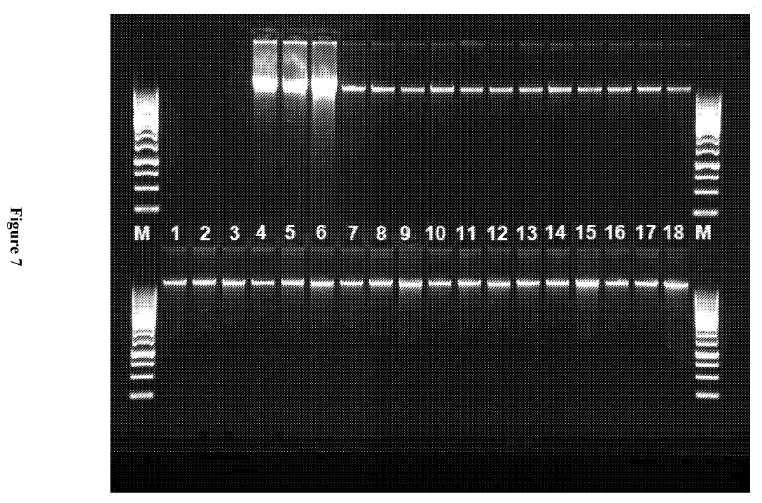

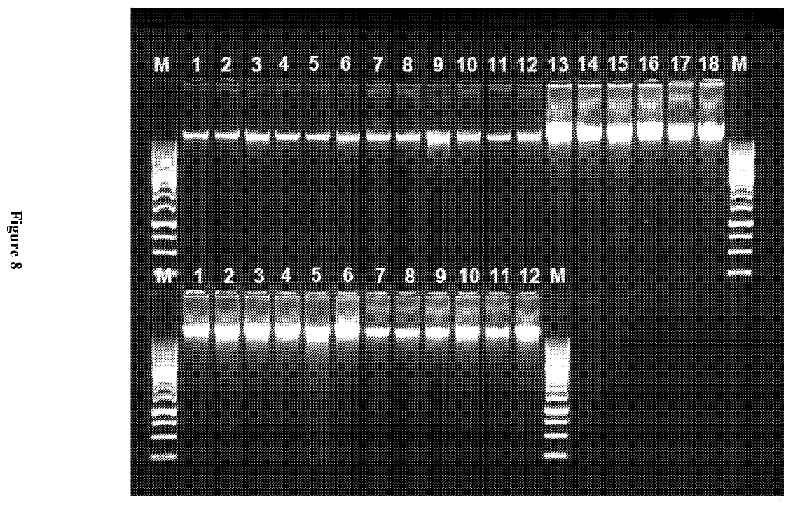

High quality gDNA was extracted from all of the samples with a visible band of gDNA seen on the agarose gel with the exception of the modified RF+ medium (FIG. 7) and a noticeable increase in the quantity of gDNA was seen in the 19 h samples (FIG. 8).

The background numbers of M. elsdenii cells were detected in the rumen fluid from steer #1990 (used for the inoculum) and the modified RF+ medium were all well below the detection cut-off of 10,000 cells/mL used for the quantitative PCR assay. The estimated M. elsdenii cell numbers are shown in Table 12.

TABLE 12
Estimated M. elsdenii cell numbers/mL in rumen fluid samples
from steer #1990 and modified RF+ medium

| Sample | M. elsdenii (cells/mL) |
|---|---|
| #1990 rumen fluid 1 | 210 |
| #1990 rumen fluid 2 | 343 |
| #1990 rumen fluid 3 | 2860 |
| Modified RF+ medium 1 | 549 |
| Modified RF+ medium 2 | 438 |
| Modified RF+ medium 3 | 771 |

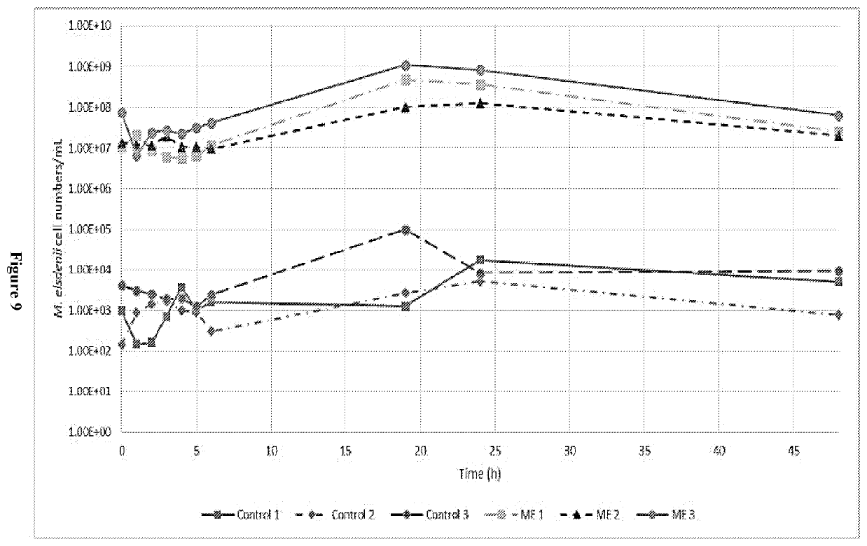

The M. elsdenii quantitative PCR was used to determine numbers of M. elsdenii cells/mL in the samples taken at each time point for each of three replicates within the Control and ME treatments (FIG. 9). At the beginning of the experiment (time 0 h) immediately after the addition of the vial of encapsulated ME, replicates ME 1 and ME 2 had 1.09×10⁷ cells/mL and 1.32×10⁷ cells/mL whilst replicate ME 3 was six times higher at 7.48×10⁷ cells/mL. The M. elsdenii population increased 41 fold in ME 1 whilst ME 2 increased 10 fold and ME 3 increased 27 fold between time 6 h and time 19 h. The Control treatment replicates were also sampled at time 0 h and the levels of M. elsdenii cells/mL were all below the cut-off threshold of 10,000 cells/mL ranging from 142 to 4,120 cells/mL. Two replicates in the Control treatment group did increase to levels above the cut-off threshold with Control 3 reaching 9.84×10⁴ cells/mL at 19 h and Control 1 reaching 1.73×10⁴ cells/mL at 24 h.

The highest number of M. elsdenii cells were detected in the 19 h samples for the ME treatment with the populations decreasing in the 24 h and 48 h samples. This may be due to the exhaustion of substrate and accumulation of toxic by-products within the closed batch cultures. Due to the overnight incubation of the cultures, it is possible the peak of M. elsdenii cell growth may have occurred prior to the 19 h sample.

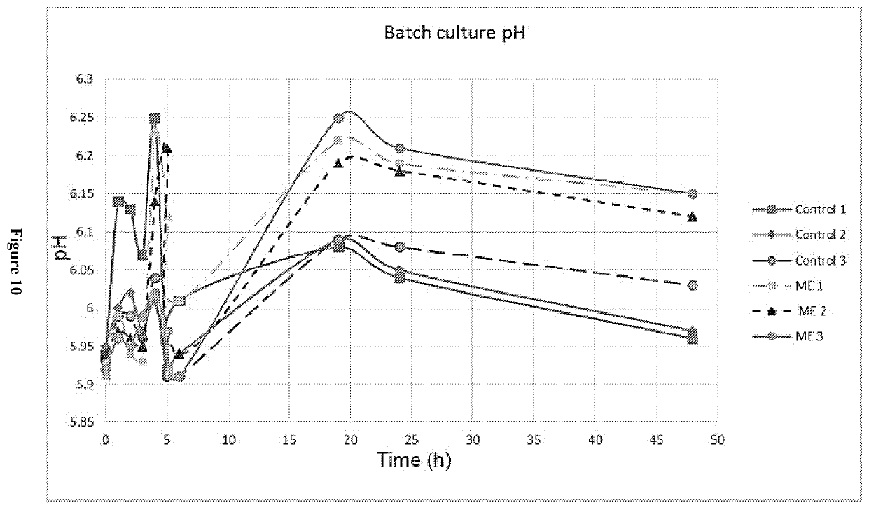

The pH of the rumen fluid, measured just prior to its addition into the Wheaton bottles, was 6.04 and the pH of the modified RF+ medium was 5.84. The pH of each of the batch cultures was measured when samples were taken (FIG. 10). The pH of the Control replicates did not drop to acute acidosis level (pH<5.0) or subacute acidosis levels (pH<5.8) but were seen to be lower than the M. elsdenii treatment groups from 19 h onwards (FIG. 10).

This experiment has successfully demonstrated that the freeze dried encapsulated M. elsdenii contained viable cells which were released from the encapsulation material and grew in the batch cultures. The M. elsdenii populations in the M. elsdenii treatment replicates, after 19 h of incubation, were present at levels 93% higher than numbers at time 0 h and 100% higher than the populations within the Control replicates after 19 h of incubation.

### Example 9. Reduction of Transition Time in Sheep

M. elsdenii YE34 was encapsulated as outlined in Example 2 above three months prior and stored and transported at ambient temperatures.

14 Merino weather lambs were selected for the trial with a random draft resulting in seven animals identified for the control and seven animals selected for the treatment group. The feeding program is shown in Table 13 and the diets fed to the animals are shown in Table 14.

### Control

The Control group was assigned to be managed in a typical transition method. Over an eight-day period lambs were slowly transition across to feedlot diet.

### Treatment

The assigned treatment group all received a capsule containing 5×10⁷ CFU of encapsulated and freeze-dried M. elsdenii YE34. These animals were then directly introduced to adlib feedlot ration.

TABLE 13
Feeding program

| Time | | Feed conditions |
|---|---|---|
| Day 1 | AM | Trail feed 50 g/hd of diet |
| | | Provide access to adlib cereal hay |
| | | Provide access to cool clean water |
| | PM | Trail feed 75 g/hd of diet |
| | | Provide access to adlib cereal hay |
| | | Provide access to cool clean water |
| Day 2 | AM | Trail feed 75 g/hd of diet |
| | | Provide access to adlib cereal hay |
| | | Provide access to cool clean water |
| | PM | Trail feed 100 g/hd of diet |
| | | Provide access to adlib cereal hay |
| | | Provide access to cool clean water |
| Day 3 | AM | Trail feed 150 g/hd of diet |
| | | Provide access to adlib cereal hay |
| | | Provide access to cool clean water |
| | PM | Trail feed 150 g/hd of diet |
| | | Provide access to adlib cereal hay |
| | | Provide access to cool clean water |
| Day 4 | AM | Trail feed 200 g/hd of diet |
| | | Provide access to adlib cereal hay |
| | | Provide access to cool clean water |
| | PM | Trail feed 200 g/hd of diet |
| | | Provide access to adlib cereal hay |
| | | Provide access to cool clean water |
| | | If all animals are transitioning onto grain well, |
| | | begin providing 50% of |
| | | the trail feed diet into the trays of self-feeders |
| Day 5 | AM | Trail feed 250 g/hd of diet |
| | | Provide access to adlib cereal hay |
| | | Provide access to cool clean water |
| | PM | Trail feed 250 g/hd of diet |
| | | Provide access to adlib cereal hay |
| | | Provide access to cool clean water |
| | | Provide 50% of the trail feed diet |
| | | into the trays of self-feeders |
| Day 6 | AM | Trail feed 300 g/hd of diet |
| | | Provide access to adlib cereal hay |
| | | Provide access to cool clean water |
| | PM | Trail feed 300 g/hd of diet |
| | | Provide access to adlib cereal hay |
| | | Provide access to cool clean water |
| | | Provide 50% of the trail feed diet |
| | | into the trays of self-feeders |
| Day 7 | AM | Trail feed 350 g/hd of diet |
| | | Provide access to adlib cereal hay |
| | | Provide access to cool clean water |
| | PM | Trail feed 350 g/hd of diet |
| | | Provide access to adlib cereal hay |
| | | Provide access to cool clean water |
| | | Provide 75% of the trail feed diet |
| | | into the trays of self-feeders |
| Day 8 | AM | Trail feed 400 g/hd of diet |
| | | Provide access to adlib cereal hay |
| | | Provide access to cool clean water |
| | PM | Trail feed 400 g/hd of diet |
| | | Provide access to adlib cereal hay |
| | | Provide access to cool clean water |
| | | Provide 75% of the trail feed diet |
| | | into the trays of self-feeders |
| Day 9 | AM | Trail feed 500 g/hd of diet |
| | | Provide access to adlib cereal hay |
| | | Provide access to cool clean water |
| | PM | Trail feed 500 g/hd of diet |
| | | Provide access to adlib cereal hay |
| | | Provide access to cool clean water |
| | | Provide 75% of the trail feed diet |
| | | into the trays of self-feeders |

TABLE 14
Diets

| | Control | |
|---|---|---|
| | Reducing Volumes | |
| Cereal Hay | over 11 days | Treatment |
| Whole Barley Grain | 75% | 75% |
| Whole Lupin Grain | 20% | 20% |
| ProAgni Protect S | 5% | 5% |
| ME Capsule | | 1 Dose × |
| | | 5 × 10⁷ CFU |

### Data Collection

Animals were weighed at the start (day 0) post five days and then post 15 days from treatment to assess the change in body weight over the induction period. Also, visual assessment was made daily on the faecal score of both the treatment and control groups to give an indication of potential lactic acidosis risk.

### Results

Table 15 shows the change in live weight for each animal throughout the transition period and Table 16 shows the faecal scores.

TABLE 15
Change in live weight

| | Day +5 | Day +15 | Total Change |
|---|---|---|---|
| Control | 3.5 | −1 | 2.5 |
| | −5 | −1 | −6 |
| | 5 | −3 | 2 |
| | −4.5 | 0.5 | −4 |
| | −1.5 | 1.5 | 0 |
| | −3.5 | 3.5 | 0 |
| | −2 | 4 | 2 |
| Average | −1.14 | 0.64 | −0.5 |
| Treatment* | −2 | 4.5 | 2.5 |
| | −1.5 | 3.5 | 2 |
| | 1 | 0.5 | 1.5 |
| | −1.5 | 1.5 | 0 |
| | 2.5 | 4 | 6.5 |
| | 0.5 | 4 | 4.5 |
| Average | −1 | 3 | 2.83 |

*one animal was excluded from the treatment group due to misadventure during the handling process

TABLE 16
Faecal scores (pen average estimates)

| | Day | Day | Day | Day | Day | Day | Day |
|---|---|---|---|---|---|---|---|
| | 1 | 2 | 3 | 4 | 5 | 6 | 7 |
| Treatment | 5 | 4 | 5 | 4 | 4 | 4 | 4 |
| Control | 2 | 3 | 3 | 3 | 3 | 4 | 4 |

Both the control group and the treatment group were successfully transitioned onto a high concentrate diet with and little to no visual signs of digestive upset or ruminal acidosis.

There was a significant difference in the time taken (10 days) for each group to reach the full intake of finisher ration, with the treatment group on full grain ration from Day 1, and the control group transitioning over 10 days.

The inclusion of a dose of encapsulated M. elsdenii YE34 during the transition process has played a significant role in the rapid adaption of the rumen microflora to a high concentrate diet without any clinical symptoms of acute lactic acidosis.

In addition, the rapid adaption to high concentrate diet has demonstrated weight gain through induction. The control group showed an average weight loss of 0.5 kg over the first 15 days of transition (Table 15). The treated animals have exhibited a gain of 2.83 kg per head, resulting in a total of +3.33 kg differential over the first 15 days of the feeding period (Table 15).

The inventors have clearly demonstrated that a single dose of 5×10⁷ CFU M. elsdenii YE34 delivered in a microencapsulated freeze dried form, has facilitated rapid and sudden transition from a grass-based diet to a high concentrate finisher diet with no impact on the health of the animal and resulted in additional weight gain compared to the control group. This rapid transition would offer significant economic returns.

### Example 10. Reduction of Transition Time in Cattle

M. elsdenii YE34 was encapsulated as outlined in Example 2 above three months prior and stored and transported at ambient temperatures.

Two trade heifers were selected to be managed with a rapid induction process from a grass based diet to a high concentrate grain diet over three days.

### Treatment

The animals received a capsule containing 6×10⁸ CFU of encapsulated and freeze dried M. elsdenii YE34. The encapsulated M. elsdenii YE34 was mixed with oil carrier in a 1 mL capsule. These animals were then transitioned over three days to an adlib feedlot ration. The feeding program is shown in Table 17 and the diet is shown in Table 18.

TABLE 17
Feeding program

| | Feed conditions |
|---|---|
| Day 1 | 1 kg Ration |
| AM | Adlib Rhodes Grass hay |
| | Provide access to cool clean water |
| PM | 1 kg ration |
| | Adlib Rhodes Grass hay |
| | Provide access to cool clean water |
| Feeding | Bunk empty, no signs scour or |
| Observation | animal health issues, cattle hungry |
| Day 2 | 2 kg Ration Adlib |
| AM | Rhodes Grass hay |
| | Provide access to cool clean water |
| PM | 2 kg ration |
| | Adlib Rhodes Grass hay |
| | Provide access to cool clean water |
| Feeding | Bunk empty, no signs scour or animal |
| Observation | health issues, cattle hungry |
| Day 3 | 3 kg ration |
| AM | Adlib Rhodes Grass hay |
| | Provide access to cool clean water |
| PM | 3 kg ration |
| | Adlib Rhodes Grass hay |
| | Provide access to cool clean water |
| Feeding | Bunk, little fibre left, no signs of scour |
| Observation | or animal health issues, cattle hungry |
| Day 4 | 7 kg Ration |
| AM | Provide access to cool clean water |
| Feeding | Bunk not empty, residual feed. Feed intake |
| Observation | maxed out at approx 2.8% bodyweight. |
| | No signs of scour or ill health. |

TABLE 18
Diet

| | Treatment |
|---|---|
| Cracked Wheat Grain | 20% |
| Cracked Lupin Grain | 8% |
| Cracked Maize grain | 46.67% |
| ProAgni Protect C | 5% |
| Urea | 0.33% |
| Rhodes Grass hay | 20% |
| ME YE34 Capsule | 1 Dose × 6 × 10⁸ CFU |
| | in 1 ml capsule, |
| | with oil carrier. |

### Data Collection

Animals were weighed at the start (day 0) post five days and then post 10 days from treatment to assess the change in body weight over the induction period. Also, visual assessment was made daily on the faecal score of both the treatment and control groups to give an indication of potential lactic acidosis risk.

### Results

Table 19 shows the change in live weight for each animal throughout the transition period and Table 20 shows the faecal scores.

TABLE 19
Change in live weight

| | Start | Day 10 |
|---|---|---|
| Animal 1 | 192 kg | 202 kg |
| Animal 2 | 200 kg | 212 kg |

TABLE 20
Faecal scores (pen average estimates)

| | Day | Day | Day | Day | Day | Day | Day |
|---|---|---|---|---|---|---|---|
| | 1 | 2 | 3 | 4 | 5 | 6 | 7 |
| Treatment | 5 | 4 | 5 | 4 | 4 | 4 | 4 |

Both the animals were successfully transitioned onto a high concentrate diet with minimal fibre and little to no visual signs of digestive upset or ruminal acidosis. The inclusion of an encapsulated dose of M. elsdenii YE34 in the transition process has played a significant role in the rapid adaption of the rumen microflora to a high concentrate diet without any clinical symptoms of acute lactic acidosis.

The rapid adaption to high concentrate diet has demonstrated weight gain through induction, which would offer significant economic returns. The treated animals have exhibited a gain of 1 kg per head per day in the first 10 days of transition when typically weight loss would have been observed in this period.

The inventors have demonstrated that a single dose of a capsule containing 6×10⁸ CFU of encapsulated and freeze-dried M. elsdenii YE34, has facilitated rapid and sudden transition from a grass-based diet to a high concentrate finisher diet with no impact on the health of the animal and animal weight gain.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A capsule comprising one or more strains of strict obligate anaerobic bacteria, wherein the capsule has a porous wall comprising surface pores with a molecular weight cut off between 50 and 200 kDa, wherein the porous wall comprises a complex formed from sodium cellulose sulphate and poly[dimethyldially-ammonium chloride].

2. The capsule of claim 1, wherein the one or more strains of strict obligate anaerobic bacteria encapsulated in the capsule have improved stability in the presence of oxygen relative to a corresponding one or more strains of the bacteria not encapsulated in the capsule and/or wherein the capsule is freeze-dried.

3. (canceled)

4. The capsule of claim 1, wherein at least one of the strains of strict obligate anaerobic bacteria is:
i) a lactic acid-utilising bacteria (LUB) or a starch-utilising bacteria;
ii) from a genus selected Megasphaera and/or Ruminococcus; and/or
iii) Megasphaera elsdenii or Ruminococcus bromii.

5-7. (canceled)

8. The capsule of claim 1 comprising Megasphaera elsdenii and Ruminococcus bromii and/or wherein the capsule does not comprise bacteria from a genus selected from the group consisting of: Bifidobacterium, Bacteroides, Fusobacterium, Propionibacterium, Enterococcus, Lactococcus, Peptostrepococcus, Pediococcus, Leuconostoc, Weissella, Geobacillus, and Lactobacillus.

9. (canceled)

10. The capsule of claim 1, wherein:
i) the porous capsule contains at least 0.2×10⁶ CFU of the strict obligate anaerobic bacteria; and/or
ii) the bacteria are suspended in the log phase of growth within the capsule; and/or
iii) the one or more strains of strict obligate anaerobic bacteria remain viable within the capsule for at least 30 days when stored under anaerobic conditions at ambient temperatures.

11-12. (canceled)

13. The capsule of claim 10, wherein:
i) the porous capsule contains at least about 0.4×10⁵ CFU of the strict obligate anaerobic bacteria one month after encapsulation following storage under anaerobic conditions at ambient temperature; or
ii) the porous capsule contains at least about 0.4×10⁵ CFU of the strict obligate anaerobic bacteria three months after encapsulation following storage under anaerobic conditions at ambient temperature; or
iii) the porous capsule contains at least about 0.2×10⁵ CFU of the strict obligate anaerobic bacteria eight months after encapsulation following storage under anaerobic conditions at ambient temperature.

14-15. (canceled)

16. The capsule of claim 13, wherein the capsule has been stored at an ambient temperature of up to about 35° C.

17. A composition comprising one or more capsules of claim 1.

18. The composition of claim 17, wherein:
i) the composition comprises one or more carriers with low oxygen diffusion rate; and/or
ii) the composition is an animal feed additive; and/or
iii) the composition is packaged in a container under anaerobic conditions.

19. (canceled)

20. The composition of claim 18 provided in a dry form or a liquid form.

21. (canceled)

22. The composition of claim 17, where said composition is provided in a dosage form comprising at least about 1×10⁶ CFU of the at least one strict obligate anaerobic bacteria.

23. (canceled)

24. A method of increasing a population of a strict obligate anaerobic bacteria in the gastrointestinal tract of an animal, comprising administering the capsule of claim 1 or the composition of claim 18 to the animal, optionally wherein at least about 1×10⁶ CFU of the strict obligate anaerobic bacteria is administered to the gastrointestinal tract of the animal.

25. The method of claim 24, wherein increasing the population of the strict obligate anaerobic bacteria in the gastrointestinal tract of the animal achieves one or more of the following:
(i) facilitates adaptation of the animal to a diet having a relatively higher amount of fermentable carbohydrates;
(ii) reduces accumulation of organic acid in the gastrointestinal tract of the animal;
(iii) improves starch utilisation in the gastrointestinal tract of the animal;
(iv) prevents or treats lactic acidosis, or one or more associated conditions or clinical symptoms thereof, in the animal;
(v) induces satiety in the animal;
(vi) controls or modulates food intake by the animal;
(vii) improves lactate utilization in the gastrointestinal tract of the animal;
(viii) improves one or more production traits selected from milk production, feedlot performance, growth rate, rate of gain, finishing time and feed conversion efficiency;
(ix) lowers the chance of digestive morbidity and/or mortality of the animal; and/or
(x) improves tolerance of an animal to a diet which is relatively higher in fermentable carbohydrates and/or lower in structural carbohydrates, without development of lactic acidosis.

26. The method of claim 25, wherein:
the condition associated with lactic acidosis is selected from the group consisting of rumenitis, lactic acidosis induced laminitis, lactic acidosis induced bloat, polioencaphomelacia (PEM), colic, gastric ulcers, dehydration and liver abscesses; or
the one or more clinical symptoms of lactic acidosis are selected from reduced feed intake, reduced feed-conversion efficiency, weight loss, lameness, diarrhea, dehydration, reduced physical performance, slow recovery from exercise, crib-biting, wind-sucking and weaving behaviour.

27. (canceled)

28. The method of claim 25, wherein:
the acidosis is acute acidosis or subacute acidosis; and/or
administration of the capsule or composition comprising same to the gastrointestinal tract of the animal maintains a stable pH in the gastrointestinal tract of the animal.

29. (canceled)

30. The method of claim 25, wherein:
(i) the animal is a ruminant and administration of the capsule or composition comprising same to the gastrointestinal tract of the animal increases pH of the rumen and/or maintains pH of the rumen above about 5.5, optionally wherein the ruminant is selected from the group consisting of sheep, cattle, goats, buffaloes, deer and camels; or
(ii) the animal is a hindgut fermenter and administration of the capsule or composition comprising same to the gastrointestinal tract of the animal increases pH of the hindgut and/or maintains pH of the hindgut above about 5.5, optionally wherein the hindgut fermenter is a horse.

31-34. (canceled)

35. A method of improving the stability of a strict obligate anaerobic bacteria, said method comprising encapsulating the strict obligate anaerobic bacteria in a capsule having a porous wall comprising surface pores with a molecular weight cut off between 50 and 200 kDa, wherein the porous wall comprises a complex formed from sodium cellulose sulphate and poly[dimethyldially-ammonium chloride].

36. The method of claim 35, wherein:
i) the capsule is a capsule of claim 1 and the strict obligate anaerobic bacteria is a strict obligate anaerobic bacteria comprised in the capsule of claim 1; and/or
ii) the one or more strains of strict obligate anaerobic bacteria remain viable within the capsule at least about 30 days post encapsulation, or at least about 2 months post encapsulation, or at least about 3 months post encapsulation, or at least about 4 months post encapsulation, or at least about 5 months post encapsulation, or at least about 6 months post encapsulation, or at least about 7 months post encapsulation, or at least about 8 months post encapsulation, when the capsule is stored under anaerobic conditions at ambient temperatures; and/or
iii) the one or more strains of strict obligate anaerobic bacteria remain viable within the capsule for at least about 30 minutes, or at least about 1 hour, or at least about 90 minutes, or at least about 2 hours, or at least about 3 hours when exposed to atmospheric oxygen levels.

37-38. (canceled)

39. A method of producing a capsule of claim 1, the method comprising:
(i) culturing one or more strict obligate anaerobic bacteria in an anaerobic media;
(ii) encapsulating the bacteria in a capsule having a porous wall comprising surface pores with a molecular weight cut off between 50 and 200 kDa, wherein the porous wall comprises a complex formed from sodium cellulose sulphate and poly[dimethyldially-ammonium chloride]; and
(iii) performing one or more step which suspend growth of bacteria;
and optionally cultivating the encapsulated bacteria in an anaerobic media prior to performing step (iii).

40. The method of claim 39, wherein:
i) the strict obligate anaerobic bacteria is a lactic acid utilising bacteria and the anaerobic media is a MRS media; or
the strict obligate anaerobic bacteria is a starch utilising bacteria and the anaerobic media is a maltose media, optionally containing rumen fluid; and/or
ii) the one or more step which suspend the growth of bacteria comprises any one of freeze-drying, spray-drying or extrusion.

41. (canceled)
